SEQUENCE LISTING (18 sequences)

SEQ ID NO: 1 - protein, 124 aa, Vibrio cholerae
MIKLKFGVFFTVLLSSAYAHGTPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMAN

SEQ ID NO: 2 - DNA, 375 bp, Vibrio cholerae
atgattaaattaaaatttggtgttttttttacagttttactatcttcagcatatgcacatggaacacctcaaaatattactgatttgtgtgcagaataccacaacacacaaatacatacgctaaatgataagatattttcgtatacagaatctctagctggaaaaagagagatggctatcattacttttaagaatggtgcaacttttcaagtagaagtaccaggtagtcaacatatagattcacaaaaaaaagcgattgaaaggatgaaggataccctgaggattgcatatcttactgaagctaaagtcgaaaagttatgtgtatggaataataaaacgcctcatgcgattgccgcaattagtatggcaaattaa

SEQ ID NO: 3 - protein, 22 aa, Escherichia coli
MNKVKCYVLFTALLSSLYAHGA

SEQ ID NO: 4 - DNA, 66 bp, Escherichia coli
atgaataaagtaaaatgttatgttttatttacggcgttactatcctctctatatgcccatggagct

SEQ ID NO: 5 - protein, 5 aa, Simian virus 40
KKKRK

SEQ ID NO: 6 - protein, 10 aa, Influenza
AAFEDLRVLS

SEQ ID NO: 7 - protein, 5 aa, Adenovirus
KRPRP

SEQ ID NO: 8 - protein, 5 aa, Saccharomyces cerevisiae
KIPIK

SEQ ID NO: 9 - protein, 4 aa, Saccharomyces cerevisiae
PRKR

SEQ ID NO: 10 - protein, 4 aa, Phaseolus vulgaris
KDEL

SEQ ID NO: 11 - protein, 19 aa, Mus musculus
MKWVTFLLLLFISGSAFSR

SEQ ID NO: 12 - protein, 23 aa, Mus musculus
MDMRAPAQIFGFLLLLFPGTRCD

SEQ ID NO: 13 - protein, 19 aa, Mus musculus
MRSLLILVLCFLPLAALGK

SEQ ID NO: 14 - protein, 22 aa, Mus musculus
MNSQVSARKAGTLLLLMMSNLL

SEQ ID NO: 15 - protein, 23 aa, Escherichia coli
MSIQHFRVALIPFFAFCLPVFAH

SEQ ID NO: 16 - protein, 17 aa, Vesicular Stomatitis Virus
MKCLLYLAFLFIHVNCK

SEQ ID NO: 17 - protein, 21 aa, Escherichia coli
MKATKLVLGAVILGSTLLAGC

SEQ ID NO: 18 - protein, 5 aa, Mus musculus
SDQRL

(19) United States
(12) Patent Application Publication
(10) Pub. No.: US 2006/0199778 A1
(43) Pub. Date: Sep. 7, 2006

(54) Methods and products related to non-viral transfection

(76) Inventors: Rutledge Ellis-Behnke, Canton, MA (US); Gerald E Schneider, Somerville, MA (US); Lisa Teather, St. Catharines (CA)

Correspondence Address: WOLF GREENFIELD & SACKS, PC, FEDERAL RESERVE PLAZA, 600 ATLANTIC AVENUE, BOSTON, MA, 02210-2206, US

(21) Appl. No.: 10/489,512

(22) PCT Filed: Sep. 19, 2002
(86) PCT No.: PCT/US02/29916

§ 371 Date: Sep. 10, 2004

Related U.S. Application Data
(60) Provisional application No. 60/323,530, filed on Sep. 19, 2001.

(51) Int. Cl. A61K 48/00; A61K 31/724; A61K 39/02

(52) U.S. Cl. 514/44; 424/235.1; 514/58

(57) ABSTRACT

The invention relates to compositions and methods for the non-viral delivery of nucleic acid molecules and/or organelles to cells in vitro and in vivo. The non-viral delivery complexes provided by the invention comprise cholera toxin B subunit or functional equivalents thereof and preferably cyclodextrin.

### RELATED APPLICATIONS

This application claims priority to U.S. Provisional Patent Application filed Sep. 19, 2001 entitled “METHODS AND PRODUCTS RELATED TO NON-VIRAL TRANSFECTION”, Ser. No. 60/323,530, the contents of which are incorporated by reference herein in their entirety.

### GOVERNMENT SUPPORT

This work was funded in part by grant number EY00126 NIH-NEI, from the National Institutes of Health. Accordingly, the United States Government may have certain rights to this invention.

### FIELD OF THE INVENTION

The invention relates to the delivery of nucleic acid molecules and/or organelles to cells in vivo and in vitro without the use of viral elements.

### BACKGROUND OF THE INVENTION

The ability to transfer nucleic acid molecules and/or organelles into cells has vast experimental and therapeutic implications. Many diseases are due to the inability to produce a certain protein. Still many others are due to the overproduction of defective (e.g., dominant negative) protein.

Many different chemical, electrochemical and biological approaches have been used to delivery nucleic acid molecules into cells. (See Table 1 and Table 2.) In vitro chemical methods include osmotic shock transformation of prokaryotic cells and calcium phosphate transfection and liposome-mediated transfer for eukaryotic cells. Nucleic acid molecules, namely DNA, have also been delivered to cells by electroporation. While this latter approach is amenable to nucleic acid molecule transfer in vitro, it is inherently unsuitable for in vivo use. Biological approaches have focused on viral strategies which include retroviral and most recently adenoviral and lentiviral mediated gene transfer into cells in culture and, in some instances, cells in vivo. A common disadvantage of the above-mentioned strategies is their inability to specifically target cells for nucleic acid molecule delivery. Targeting of cell subsets usually requires the selective harvesting of cells followed by in vitro delivery and re-introduction in vivo.

Viral mediated gene transfer requires the in vitro production of defective viral particles which encapsulate a nucleic acid molecule of a finite size. The encapsulated nucleic acid molecule, usually referred to as a viral vector, is a recombinant nucleic acid molecule which contains a gene(s) of interest cloned between 5′ and 3′ flanking viral cis elements. The cis elements are required for integration into the host genome yet they are also capable of transcriptional regulation. As a result, these elements have the potential to interfere with the transcriptional activity of the cloned gene(s). Another limitation of viral mediated gene transfer is the need for and the difficulty in achieving high titre viral stocks. In vivo infection with viruses, when applicable, is generally not effective given the in vivo dilution of viral particles. Additionally, although both retroviral and adenoviral methods employ replication-defective viral particles, the possibility of producing replication-competent viruses and thereby causing active infection in vivo is an inherent danger of both systems. Yet another Is limitation of the above-mentioned viral methods is their ability to stimulate an immune response in vivo. The resultant immune response is undesirable at least because it may be directed to the viral vector and the polypeptides produced by the viral vector.

For retroviral mediated gene transfer to occur, target cells whether in vitro or in vivo must be in a cycling status. Since retroviruses package nucleic acid molecules in the form of RNA, reverse transcription of the RNA to DNA is required for integration into the host genome from where the gene exerts its effects. Cells which divide infrequently or never at all, such as some classes of stem cells or terminally differentiated end cells, are usually less amenable to gene transfer via retroviral infection as compared to rapidly dividing cells. Thus diseases for which a long-term cure is dependent upon stem cell or end cell manipulation are poor candidates for gene therapy treatment using retroviral transfection. Retroviral use is also limited to the restricted range of host infectivity specific to each strain of virus. In contrast adenoviruses which contain double stranded DNA do not require target cells to be cycling for infection, integration and propagation.

DNA has also been delivered to cells using receptor-mediated endocytosis. In this approach, DNA is initially complexed with polycations such as polylysine for condensation and charge neutralization purposes. Ligands for cell surface receptors, such as transferrin, are then coupled either biochemically or enzymatically to the polylysine moieties. In a further modification, the transferrin molecules are coupled to the outer surface of inactivated adenoviral particles. The adenoviral particles can effect the release of the DNA/polylysine/transferrin complex from endosomes prior to lysosome mediated degradation.

In contrast to the use of polycations for complexing DNA, other approaches have incorporated specific DNA binding domains which recognize and bind distinct nucleic acid molecule consensus sequences. An example of this is the use of the GAL4 DNA binding domain of yeast which selectively binds to a 17 bp sequence. Using this approach, a nucleic acid molecule must usually be modified to incorporate artificial GAL4 binding sites. Likewise, other approaches which rely on a consensus sequence dependent DNA binding domain will similarly require modification of the transferred nucleic acid molecule.

TABLE 1
Prior Art Transfection Methods:

| | EXPRESSION | | CELL | CELL | |
|---|---|---|---|---|---|
| METHOD | Transient | Stable | TOXICITY | TYPES | COMMENTS |
| MOS Non-viral | Yes | Yes | No | Many | Non-viral encapsulant forms |
| Encapsulation | | | | | membrane around DNA; |
| | | | | | target sequence directs |
| | | | | | encapsulant to cells and is |
| | | | | | absorbed by endocytosis |
| Retroviral | Yes | Yes | No | Many (after | Virus is packaged with DNA in |
| | | | | transfection of | helper cell line and then |
| | | | | appropriate | allowed to infect cells |
| | | | | receptor) | |
| Lipofection | Yes | Yes | Varies | Adherent cells, | Cationic liposomes bind DNA |
| | | | | primary cell | and then fuse with the |
| | | | | lines, | negatively charged cell |
| | | | | suspension | membrane |
| | | | | cultures | |
| CaPO₄— | Yes | Yes | No | Adherent cells, | Calcium phosphate |
| Precipitation | | | | suspension | coprecipitates with DNA, |
| | | | | cultures | which is absorbed into the cell |
| | | | | | by endocytosis |
| DEAE-Dextran | Yes | No | Yes | BSC-1, CV-1, | DEAE binds to phosphate |
| | | | | and COS | groups of DNA and is |
| | | | | | believed to be absorbed by |
| | | | | | endocytosis |
| Electroporation | Yes | Yes | No | Many | Brief electric pulse forms nm- |
| | | | | | sized pores in cell membrane; |
| | | | | | DNA is taken into cytoplasm |
| | | | | | directly |
| Biolistics | Yes | Yes | No | Primary cell | Small particles of tungsten or |
| | | | | lines, tissues, | gold bind DNA and are |
| | | | | organs, and | delivered into cells via a |
| | | | | plant cells | particle accelerator system |
| Polybrene | Yes | Yes | Varies | CHO and | Polycation Polybrene allows |
| | | | | keratinocytes | the efficient and stable |
| | | | | | introduction of low molecular |
| | | | | | weight DNA into cell lines |

TABLE 2
Commercially Available Kits for Transfection

| Method | Kit/Product | Manufacturer |
|---|---|---|
| Retroviral | Retro-X | CLONTECH |
| Transduction | | |
| Electroporation | Gene Pulsar II | Bio-Rad |
| Lipofection | CytoFectene | Bio-Rad |
| | Fugene 6 | Boehringer- |
| | | Mannheim |
| | Lipofectamine, | Life Technologies |
| | LipofectAce | (Invitrogen) |
| | Perfect Lipid | |
| | LipoTaxi | Stratagene |
| CaPO₄- | CellPhect | Amersham- |
| Precipitation | | Pharmacia Biotech |
| | CellPhos Mammalian | CLONTECH |
| | Profectin | Promega |
| | MBS Mammalian | Stratagene |
| DEAE-Dextran | DEAE Transfection | 5 Prime → 3 Prime |
| | DEAE-Dextran | Sigma Aldrich |
| | Mammalian | Stratagene |
| | Transfection | |

### SUMMARY OF THE INVENTION

The invention relates to products and methods for delivering nucleic acid molecules and/or organelles of various sizes into cells either in vitro or in vivo. The invention is based on the surprising discovery that cholera toxin B subunit can deliver a nucleic acid molecule and/or an organelle to a cell such as a central nervous system cell. This is a surprising finding since cholera toxin has been used extensively as an adjuvant. It has not been heretofore recognized that cholera toxin B subunit is capable of effecting delivery of nucleic acid molecules or organelles into cells in vitro or in vivo. The invention is further based on the observation that addition of cyclodextrin further increases the efficiency of delivery into recipient cells.

The invention provides compositions and methods of use relating to cholera toxin B subunit, optionally in the presence of cyclodextrin, for the delivery of nucleic acid molecules and/or organelles in vivo, ex vivo and in vitro.

In one aspect, the invention provides a non-viral biological molecule delivery complexes. These complexes can be used to deliver, inter alia, nucleic acid molecules and/or organelles to cells in vitro and in vivo.

In one aspect, the invention provides a non-viral nucleic acid delivery complex or a non-viral organelle delivery complex comprising a cyclodextrin, and a cholera toxin B subunit or functional equivalent thereof.

In one embodiment, the cyclodextrin is selected from the group consisting of α-cyclodextrin, β-cyclodextrin, and γ-cyclodextrin. Preferably, wherein the cyclodextrin is γ-cyclodextrin. The foregoing embodiments for cyclodextrin apply equally to all aspects of the invention.

In another embodiment, the cholera toxin B subunit or functional equivalent thereof is selected from the group consisting of cholera toxin B subunit and E. coli enterotoxin. Preferably, the cholera toxin B subunit or functional equivalent thereof is cholera toxin B subunit. In other embodiments, the cholera toxin B subunit or functional equivalent thereof is a pore or a channel insertion complex. In related embodiments, the channel insertion complex may be a chloride channel, potassium channel, sodium channel, calcium channel, or an antiport. The foregoing embodiments for cholera toxin B subunit or functional equivalent thereof apply equally to all aspects of the invention.

In embodiments in which the delivery complex is a nucleic acid delivery complex, it may further comprise a nucleic acid molecule. In one embodiment, the nucleic acid molecule encodes a polypeptide. In another embodiment, the nucleic acid molecule is an antisense nucleic acid molecule. In yet another embodiment, the nucleic acid molecule is a DNA molecule. Alternatively, the nucleic acid molecule is an RNA molecule. The nucleic acid molecule may also be a DNA/RNA hybrid molecule. In important embodiments, the nucleic acid molecule encodes a polypeptide selected from the group consisting of a cystic fibrosis transmembrane conductance regulator (CFTR), a Bcl-2 polypeptide, IFκB (i.e., IκB), A-beta regulatory protein, parkin, dystrophin, thymidine kinase, cytosine deaminase, nerve growth factor, apolipoprotein, E ε4, huntingtin, APP, p53, Rb, WT-1, PDGF, lis-1, BDNF, FDGF, IL-12 p40, protease inhibitors and IL-4 (which can be used to inhibit Thl induction and macrophage activation). In one preferred embodiment, the nucleic acid molecule encodes a cystic fibrosis transmembrane conductance regulator (CFTR). In still other embodiments, the nucleic acid molecule comprises a regulatory sequence. The regulatory sequence may be selected from the group consisting of a promoter, an enhancer, an intron, a 3′ untranslated region, and a 5′ untranslated region, but is not so limited. The foregoing embodiments for a nucleic acid molecule apply equally to all aspects of the invention.

In embodiments in which the delivery complex is an organelle delivery complex, it may further comprise an organelle. The organelle can be selected from the group consisting of a mitochondrion, a nucleus, a Golgi apparatus, an endoplasmic reticulum, a chloroplast, a lysosome, a plastid, but is not so limited.

In still other embodiments, the complex further comprises an organelle targeting compound or a cytoskeleton targeting compound. The organelle targeting compound may be a compound that targets the complex (and/or the nucleic acid molecule, if present) to an organelle such as a nucleus, a mitochondria, an endoplasmic reticulum, or a chloroplast. The cytoskeleton targeting compound may be a cytoskeleton polypeptide or a fragment thereof selected from the group consisting of actin, myosin, spectrin, kinesin, dynine, heavy, medium, light, neurofilament proteins and Gap-43. The foregoing embodiments for an organelle targeting compound or a cytoskeleton targeting apply equally to all aspects of the invention.

In another aspect, the invention provides a pharmaceutical composition comprising a therapeutically or prophylactically effective amount of any of the non-viral nucleic acid delivery or organelle delivery complexes described above, and a pharmaceutically acceptable carrier. In one embodiment, the pharmaceutical composition further comprises a nucleic acid molecule or an organelle depending upon what the delivery complex is intended to deliver.

In a related aspect, the invention provides a kit comprising the pharmaceutical composition described above, and instructions for use thereof.

In one aspect, the invention provides a method for delivering a nucleic acid molecule into a cell comprising delivering to a cell a nucleic acid molecule complexed with a cholera toxin B subunit or functional equivalent thereof, in an amount effective to deliver the nucleic acid molecule into the cell.

In a related aspect, the invention provides a method for delivering an organelle into a cell comprising delivering to a cell organelle complexed with a cholera toxin B subunit or functional equivalent thereof, in an amount effective to deliver the organelle into the cell.

In one embodiment, the cell is a eukaryotic cell. The cell may alternatively be a prokaryotic cell. In yet another embodiment, the cell is selected from the group consisting of an animal cell, a human cell, an insect cell and a plant cell. In related embodiments, the animal cell may be a mouse cell, and the insect cell may be a Drosophila cell. In important embodiments, the cell is a central nervous system cell.

In certain embodiments, the cell is present in a subject or is harvested from a subject. The cell may be in a suspension, a tissue or fragment thereof, or an organ or fragment thereof, but is not so limited. In other embodiments, the subject from which the cell is harvested has or is at risk of developing a disorder selected from the group consisting of cystic fibrosis, muscular dystrophy, Alzheimer's disease, Parkinson's disease, arthritis, neurodegenerative disorders, retinal degenerative disorders, cancers, Usher syndrome, Down Syndrome, multiple sclerosis (MS), ALS, metabolic disorders, and lysocephaly. In one embodiment, the cell is present in or harvested from a subject known to have or at risk of having one or more genetic mutations. According to one embodiment, the nucleic acid molecule or the organelle is delivered to the cell by passive or active transport.

In one embodiment, the method further comprises complexing the nucleic acid molecule (or the organelle) and the cholera toxin B subunit or functional equivalent thereof with a cyclodextrin. In a related embodiment, the nucleic acid molecule (or the organelle) complexed with a cholera toxin B subunit or functional equivalent thereof and cyclodextrin is delivered to a subject via a route selected from the group consisting of dermal, transdermal, inhalation, ocular, injection, and orally. In yet another embodiment, the method further comprises complexing the nucleic acid molecule (or the organelle) and the cholera toxin B subunit or functional equivalent thereof with an organelle targeting compound or a cytoskeleton targeting compound.

In another aspect, the invention provides a composition comprising a cholera toxin B subunit or functional equivalent thereof and a nucleic acid molecule, or a cholera toxin B or functional equivalent thereof and an organelle. In one embodiment, the composition further comprises a cyclodextrin.

In yet another aspect, the invention provides a method for producing a non-viral nucleic acid delivery composition comprising combining a cholera toxin B subunit or a functional equivalent thereof, and a nucleic acid molecule. In an important embodiment, the nucleic acid molecule encodes a polypeptide. In other embodiments, the cholera toxin B subunit or functional equivalent thereof is present in amount effective to deliver the nucleic acid molecule to a cell, provided that amount is not capable of inducing an adjuvant effect.

In one embodiment, the method further comprises combining the cholera toxin B subunit or a functional equivalent thereof and the nucleic acid molecule with a cyclodextrin.

In a related embodiment, the cyclodextrin and the nucleic acid molecule are first combined.

In a related aspect, the invention provides a method for producing a non-viral organelle delivery composition comprising combining a cholera toxin B subunit or a functional equivalent thereof, and an organelle. In other embodiments, the cholera toxin B subunit or functional equivalent thereof is present in amount effective to deliver the organelle to a cell, provided that amount is not capable of inducing an adjuvant effect.

In one embodiment, the method further comprises combining the cholera toxin B subunit or a functional equivalent thereof and the organelle with a cyclodextrin. In a related embodiment, the cyclodextrin and the organelle are first combined.

In yet another aspect, the invention provides a method for promoting cell survival in vivo or in vitro. The method comprises contacting a cell with an effective amount of an anti-apoptotic agent in a non-viral delivery complex comprising a cholera toxin B subunit or functional equivalent thereof for a time sufficient to transfect the cell. As used herein, an effective amount of an anti-apoptotic agent is an amount sufficient to inhibit apoptosis or the phenotypic alterations associated with apoptosis (e.g., macroscopic alterations such as membrane blebbing, or molecular changes such as upregulation or downregulation of particular transcripts). The effective amount can be determined by analysis of cell death rate. As used herein, an anti-apoptotic agent is an agent that inhibits apoptosis. Such an agent can be a small organic or inorganic molecule. It may also be nucleic acid or peptide in nature.

In one embodiment, the anti-apoptotic agent is selected from the group consisting of caspase inhibitor, β-cytokine, IL-9, Bcl-2, Bcl-X_{L}, fink, Islet-Brain 1 (IB 1), p35, crmA, Mcl-1, E1B-19K, Bm-3a, Xiap, IGF-1, Hsp27, ILP, anti-Fas agent, and anti-Fas ligand agent. The β-cytokine may be selected from the group consisting of P500 and TCA-3/I-309, but it is not so limited. In another embodiment, the anti-apoptotic agent is selected from the group consisting of LiCl, glutathione peroxidase 1, and aurintricarboxylic acid. In still another embodiment, the anti-apoptotic agent is a neurotrophic factor. Those of ordinary skill in the art will be familiar with other anti-apoptotic agents that can be used in this method.

In one embodiment, the cell is a central nervous system cell. For example, the cell may be a neuron or a retinal ganglion, but it is not so limited.

In other embodiments, the contacting occurs in vivo, and may comprise injection of the anti-apoptotic agent into a caudal superior colliculus. In still other embodiments, the contacting occurs following injury to the cell.

The embodiments recited above with respect to other aspects of the invention apply equally to this aspect. For example, the delivery complex further may further comprise a cyclodextrin. The cyclodextrin may be selected from the group consisting of α-cyclodextrin, β-cyclodextrin, and γ-cyclodextrin. In important embodiments, the cyclodextrin is γ-cyclodextrin. In other embodiments, the cholera toxin B subunit or functional equivalent thereof is cholera toxin B subunit.

Each of the limitations of the invention can encompass various embodiments of the invention. It is therefore anticipated that each of the limitations of the invention involving any one element or combinations of elements can be included in each aspect of the invention.

### BRIEF DESCRIPTION OF THE SEQUENCE LISTING

SEQ ID NO:1 is the amino acid sequence of cholera toxin B subunit (GenBank Accession No. U25679.

SEQ ID NO:2 is the nucleotide sequence of cholera toxin B subunit (GenBank Accession No. U25679.

SEQ ID NO:3 is the amino acid sequence of enterotoxin subunit B (GenBank Accession No. M17101.

SEQ ID NO:4 is the nucleotide sequence of enterotoxin subunit B (GenBank Accession No. M17101.

SEQ ID NO:5 is the amino acid sequence of the nuclear leader sequence of the simian virus SV40 large tumor antigen.

SEQ ID NO:6 is the amino acid sequence of the NLS from influenza virus nucleoprotein C-terminal residues 336-345.

SEQ ID NO:7 is the amino acid sequence of the NLS from adenovirus E1a C-terminal amino acids.

SEQ ID NO:8 is the amino acid sequence of the NLS from yeast mata2 residues 1-13.

SEQ ID NO:9 is the amino acid sequence of the NLS from yeast ribosomal protein L3 residues 1-21.

SEQ ID NO:10 is the amino acid sequence of an endoplasmic reticulum sequence.

SEQ ID NO:11 is the amino acid sequence of a signal peptide from proproalbumin.

SEQ ID NO:12 is the amino acid sequence of a signal peptide from pre-IgG light chain.

SEQ ID NO:13 is the amino acid sequence of a signal peptide from prelysozyme.

SEQ ID NO:14 is the amino acid sequence of a signal peptide from preprolactin.

SEQ ID NO:15 is the amino acid sequence of a signal peptide from prepenicillinase.

SEQ ID NO:16 is the amino acid sequence of a signal peptide from prevesicular stomatitis virus (VSV) glycoprotein.

SEQ ID NO:17 is the amino acid sequence of a signal peptide from prelipoprotein.

SEQ ID NO:18 is the amino acid sequence of a trans-Golgi network sequence.

### BRIEF DESCRIPTION OF THE DRAWINGS

FIG. 1 is an injection diagram showing the dorsal view of the hamster brain with cortex removed (rostral is up and caudal is down). The sites of injection are in the caudal midbrain tectum. Legend: SC: superior colliculus; PT: pre tectum; LP: lateral posterior; MGB: medial geniculate body; IC: inferior colliculus.

FIG. 2 is a photograph of Bcl-2 protein expression in the zona inserta (on the side ipsilateral of the injection) following transfection by a Bcl-2 plasmid into live animals. The protein Bcl-2 was visualized by immunohistochemistry using an anti-Bcl-2 antibody and a secondary antibody conjugated to an Alexam fluorescent dye (red). Scale bar is 100 μm.

FIG. 3 is a photograph showing the control (contralateral substantia nigra; bottom panel) and test area (substantia nigra; top panel) of the transfected animal. The protein Bcl-2 was visualized by immunohistochemistry using an anti-Bcl-2 antibody and a secondary antibody conjugated to an Alexa™ 546 fluorescent dye (Molecular Probes, Inc.).

FIG. 4 is a photograph of neurons of the midbrain central gray showing Bcl-2 protein that was produced after the transfection with a Bcl-2 plasmid. Note the outlines of some neurons and their nuclei drawn with bright-field microscopy with a reduced aperture to increase contrast. The protein is localized in the cytoplasm. Scale bar is 50 μm.

FIG. 5 is a photograph that shows the transfection of EGFP gene in explanted mouse retinal tissue and the resultant production of EGFP in green (top panel). The bottom panel is the control that was transfected with an encapsulant with an “empty plasmid,” containing no EGFP gene. Both pictures are from the tissue fixed with 2% paraformaldehyde 24 hr after transfection. Retinal explants are from adult mice.

FIG. 6 is a photograph that shows the transfection of EGFP gene in explanted retinal tissue and the resultant production of EGFP in green (top panel). The bottom panel is the control that was transfected with an encapsulant with an “empty plasmid,” containing no EGFP gene. Both pictures are from the tissue fixed with 2% paraformaldehyde 48 hr after transfection. Retinal explants are from adult mice.

FIG. 7 is a photograph that shows the transfection of EGFP gene in explanted retinal tissue and the resultant production of EGFP in green (top panel). The bottom panel is the control that was transfected with an encapsulant with an “empty plasmid,” containing no EGFP gene. Both pictures are from the tissue fixed with 2% paraformaldehyde 72 hr after transfection. Retinal explants are from adult mice.

FIG. 8 is a photograph that shows the transfection of EGFP gene in explanted retinal tissue and the resultant production of EGFP in green (top panel). The bottom panel is the control that was transfected with an encapsulant with an “empty plasmid,” containing no EGFP gene. Both pictures are from the tissue fixed with 2% paraformaldehyde 96 hr after transfection. Retinal explants are from adult mice.

The figures provided herein are illustrative and are not required for enablement of the invention.

### DETAILED DESCRIPTION OF THE INVENTION

The invention relates, in part, to the discovery that nucleic acid molecules and/or organelles can be delivered to a cell using cholera toxin B subunit (CTB). Based on the structural and functional similarity of other membrane complexes to cholera toxin B subunit, the invention further embraces the use of functional equivalents of cholera toxin B subunit. Accordingly, the invention provides non-viral nucleic acid and/or organelle delivery complexes that comprise at a minimum a cholera toxin B subunit or a functional equivalent thereof and a nucleic acid molecule (or an organelle) as well as methods for the use of such complexes. In important embodiments, the complexes also contain a cyclodextrin.

Several aspects of the invention involve the use of cholera toxin B subunit or a functional equivalent thereof in the delivery of a nucleic acid molecule or an organelle. Cholera toxin is the major virulent factor produced by Vibrio cholerae. It is comprised of one A subunit having ADP-ribosyltransferase activity and five B subunits that function in GM1 receptor binding. Internalization of cholera toxin has been reported to include intracellular trafficking through a retrograde vesicular pathway to the trans-Golgi network, Golgi apparatus and endoplasmic reticulum. This intracellular trafficking may be altered when the cholera toxin B subunit is complexed with cyclodextrin. The ability of cholera toxin to act as an adjuvant has been established. It has not been recognized prior to the present invention that cholera toxin B subunit can deliver nucleic acid molecules to a cell. Cholera toxin B subunit can be obtained from commercial sources such as List Biological Laboratories, Inc. (Campbell, Calif.), and Calbiochem.

The enterotoxin of Escherichia coli is closely related to cholera toxin. Both toxins are able to stimulate chloride secretion by intestinal epithelial cells, thereby causing diarrhea in infected subjects. Like cholera toxin, enterotoxin is comprised of a single A subunit and five B subunits. Cholera toxin, enterotoxin and their subunits are all reportedly capable of inducing an immune response.

The ability of both cholera toxin and enterotoxin to induce the secretion of chloride from a cell suggests that other channel insertion complexes are equally effective in delivering nucleic acid molecules and/or organelles to cells. Accordingly, the invention embraces the use of other channel insertion complexes in the non-viral nucleic acid and/or organelle delivery complexes described herein. As used herein, a “channel insertion complex” is a complex that can be inserted into a cell membrane (either passively or actively) and can thereby form a channel through the membrane between the extracellular and intracellular environments. In addition, in organelles having multiple membranes, a channel insertion complex can form a channel through one or all membranes. In the case of mitochondria which have an inner and outer membrane, the complex could facilitate the insertion of a channel from the innermost space, or from the intermediate space of the organelle, or from the intracellular space. The complex may be comprised of a single protein or several proteins. Preferably, the channel insertion complex forms a channel that allows passage or traffic of ions. Accordingly, the channel insertion complex may be but is not limited to a chloride channel, potassium channel, sodium channel, calcium channel, or an antiport.

The invention embraces the use of CTB and functional equivalents thereof including enterotoxin subunit B and functional equivalents thereof, and other channel insertion complexes, in the non-viral nucleic acid and/or organelle delivery complexes of the invention.

As used herein, a “variant” of CTB is a peptide or polypeptide that contains one or more modifications to the primary amino acid sequence of CTB. Generally, a “functionally equivalent variant of CTB” is a peptide or polypeptide that retains the functional capabilities of CTB that are required for the invention. For example, the functionally equivalent variant would be capable of delivery nucleic acid molecules and/or organelles into a cell, preferably in combination with a cyclodextrin.

Functional equivalence refers to an equivalent activity (e.g., delivery of a nucleic acid molecule to a cell), however it also embraces variation in the level of such activity. For example, a functional equivalent is a variant that delivers nucleic acid molecules and/or organelles to a cell with lesser, equal, or greater efficiency than the CTB described herein, provided that the variant is still useful in the invention (i.e., it delivers nucleic acid molecules and/or organelles above background levels and is not toxic to a cell). A functionally equivalent variant of CTB useful in the invention is one that delivers nucleic acid molecules or organelles with a transfection efficiency over background (i.e., over that achievable in the absence of the CTB or a functionally equivalent variant thereof. In some instances, this will be greater than 5%, greater than 10%, greater than 20%, greater than 30%, greater than 40%, greater than 50%, greater than 60%, greater than 70%, greater than 80%, greater than 90%, or greater than 100% of the transfection efficiency disclosed herein. In some embodiments, the transfection efficiency achievable with CTB is greater than 10%, and more preferably greater than 20% (i.e., greater than 20% of all cells in a targeted population of cells is transfected). Functionally equivalent variants of enterotoxin, and other channel insertion complexes are similarly defined.

The skilled artisan will realize that conservative amino acid substitutions may be made in CTB to provide functionally equivalent variants. As used herein, a “conservative amino acid substitution” refers to an amino acid substitution which does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology. F. M. Ausubel, et al., eds., John Wiley & Sons, Inc., New York. Exemplary functionally equivalent variants of CTB include conservative amino acid substitutions of SEQ ID NO: 1. Conservative substitutions of amino acids include substitutions made amongst amino acids within the following groups: (a) M, L L, V; (b) F, Y, W; (c) K, R. H; (d) A, G; (e) S, T; (f) Q, N; and (g) E, D.

Conservative amino-acid substitutions in the amino acid sequence of CTB to produce functionally equivalent variants of CTB typically are also made by alteration of a nucleic acid molecule encoding CTB (SEQ ID NO:2). Such substitutions can be made by a variety of methods known to one of ordinary skill in the art. For example, amino acid substitutions may be made by PCR-directed mutation, site-directed mutagenesis according to the method of Kunkel (Kunkel, Proc. Nat. Acad. Sci. U.S.A. 82: 488-492, 1985), or by chemical synthesis of a gene encoding CTB. The activity of functionally equivalent fragments of CTB can be tested by cloning the gene encoding the altered CTB into a bacterial or mammalian expression vector, introducing the vector into an appropriate host cell, expressing the altered CTB, and testing for a functional capability of the CTB as disclosed herein.

Modifications that create a CTB variant are typically made to the nucleic acid molecule that encodes CTB, and can include deletions, point mutations, truncations, amino acid substitutions and addition of amino acids or non-amino acid moieties to: 1) reduce or eliminate an activity of CTB (such as an adjuvant activity); 2) enhance a property of CTB, such as protein stability in an expression system, and/or the stability of protein-protein association; 3) provide a novel activity or property to CTB, such as addition of an antigenic epitope or addition of a detectable moiety; or 4) to provide equivalent or better binding to another compound.

Mutations of a nucleic acid molecule which encodes CTB preferably preserve the amino acid reading frame of the coding sequence, and preferably do not create regions in the nucleic acid molecule that are likely to hybridize to form secondary structures, such as hairpins or loops, which can be deleterious to expression of the variant polypeptide.

Mutations can be made by selecting an amino acid substitution, or by random mutagenesis of a selected site in a nucleic acid molecule which encodes the polypeptide. Variant polypeptides are then expressed and tested for one or more activities to determine which mutation provides a variant polypeptide with the desired properties. Further mutations can be made to variants (or to non-variant CTB) which are silent as to the amino acid sequence of the polypeptide, but which provide preferred codons for translation in a particular host. The preferred codons for translation of a nucleic acid molecule in, e.g., E. coli are well known to those of ordinary skill in the art. Still other mutations can be made to the noncoding sequences of a CTB gene or cDNA clone to enhance expression of the polypeptide.

Alternatively, modifications can be made directly to the polypeptide, such as by cleavage, addition of a linker molecule, addition of a detectable moiety, such as biotin, addition of a fatty acid, and the like or addition of other domains of the modular polypeptide of the invention.

Variants can include CTB variants which are modified specifically to alter a feature of the polypeptide unrelated to its physiological activity. For example, cysteine residues can be substituted or deleted to prevent unwanted disulfide linkages. Similarly, certain amino acids can be changed to enhance expression of CTB by eliminating proteolysis by proteases in an expression system (e.g., dibasic amino acid residues in yeast expression systems in which KEX2 protease activity is present).

In particularly important aspects of the invention, the non-viral nucleic acid or organelle delivery complex further comprises a cyclodextrin. Cyclodextrin is a cyclic polysaccharide that contains naturally occurring D(+)-glucopyranose units in an α-(1,4) linkage. Common forms of cyclodextrin are α-cyclodextrins, β-cyclodextrins, and γ-cyclodextrins which respectively comprise 6, 7 and 8 glucopyranose units. Cyclodextrins have been used as solubilizing agents for pharmaceutical agents. Although not intending to be bound by any particular theory, it is postulated that the solubilizing activity derives from the toroid-like conformation of the cyclodextrin molecule and the hydrophobic cavity formed therein. The size of the cavity is directly related to the number of glucopyranose units per molecule, and as a result, the cavity diameter is estimated to be 5.2, 6.4 and 8.3 angstroms for α-, β- and γ-cyclodextrins, respectively.

The various derivative forms of cyclodextrins that can be used in the invention include Is but are not limited to hydroxypropyl-, hydroxypropyl-γ-, 2-hydroxypropyl-β-, hydroxyethyl, methyl-, sulphate-substituted, ethyl-, β-cyclodextrin polysulfate, dimethyl-β-cyclodextrin, trimethyl-β-cyclodextrin, and β-cyclodextrin polysulfate. Cyclodextrins can also be used in the form of cyclodextrin liposomes.

Various forms of cyclodextrin are commercially available from sources such as RBI (Natick, Mass.), and Sigma Chemical Co. (St. Louise, Mo.). In addition, synthesis of various cyclodextrins in described in U.S. Pat. No. 5,691,316 and published PCT Patent Application WO00/33885, published on Jun. 15, 2000. Cyclodextrins have been previously used in human subjects without serious side effects.

The compositions can be used to effect the transfer to nucleic acid molecules and/or organelles into a cell either in vivo or in vitro (including ex vivo). Studies indicate the method is not cytotoxic, unlike many prior art transfection techniques. The in vitro transfection of nucleic acid molecules into cells commonly used in research laboratories which are generally cumbersome, expensive, inefficient and time-consuming can be replaced by the simple, efficient and inexpensive method of the invention. As a result of the broad flexibility of the methods, the invention can be used in numerous applications such as gene delivery in vitro, ex vivo and in vivo in animals or other organisms.

The organelles to be delivered using the complexes of the invention include but are not limited to mitochondria, Golgi apparatus, endoplasmic reticulum, lysosomes, centrioles, and nuclei. In plant cells, the organelles can further include chloroplasts and plastids.

Organelle transfer can be used to treat disorders which are characterized by a deficiency in an organelle protein or product. For example, lysosomal deficiencies can lead to classical late infantile neuronal ceroid lipofuscinosis, lysosomal acid phosphatase deficiency can lead to lysosomal storage abnormalities in kidney and CNS, mitochondrial respiratory chain function deficiency is associated with Parkinson's disease, and mitochondrial enzyme (e.g., cytochrome C oxidase) deficiency is associated with Alzheimer's disease.

Transfer of mitochondria into oocytes or adult germ line cells can also be used to “rejuvenate” or “revitalize” the adult germ line cell. Similar approaches have been used by introducing mitochondrial DNA into human germ line cells. The invention further embraces the introduction of mitochondrial DNA into cells by transferring cytoplasts loaded with such DNA using the methods of the invention. Cytoplasts loaded with mitochondrial DNA have been described previously. (Kagawa, et al. Adv. Drug Del. Rev. 2001 49:107-119.) In still other embodiments, the methods of the invention can be used to deliver mitochondria that are loaded with nuclear DNA or homologs thereof. This would provide a novel way of effecting gene therapy by using a mitochondria as a shuttle for nuclear transcripts.

One important aspect of the invention is its broad flexibility since both the non-viral delivery complex and the nucleic acid molecule (or organelle) to be delivered can be endowed with a wide variety of properties. For example, the non-viral delivery complex can be complexed to other functional entities such as organelle targeting compounds, cytoskeleton targeting compounds, and cell targeting compounds. The complex may contain one or more of these functional entities in any combination for the practice of the invention. The association of the non-viral delivery complex, or of its individual components, with the nucleic acid molecule (or the organelle) to be delivered is preferably non-covalent and in the case of nucleic acid delivery, sequence independent as well. As used herein, the term “complex” refers to the physical association of the biological molecule of interest (e.g., the nucleic acid molecule, the organelle or the anti-apoptotic agent) with one or more of the constituents of the delivery vehicle (e.g., cholera toxin subunit, cyclodextrin, or both). In some embodiments, “complex” refers to the ionic or non-ionic (e.g., covalent) association of the afore-mentioned components. In other embodiments, all of the components of the delivery vehicle may be covalently attached to each other, while in other embodiments, only a subset of the components are covalently attached to each other.

Targeting of the non-viral nucleic acid delivery complex facilitates expression of the delivered nucleic acid molecule in a particular location. For example, diseases exist that involve mutations of genes present in mitochondrial DNA. Overproduction or mutation of some mitochondrial genes has been implicated in the development of Alzheimer's disease. In addition, some mitochondrial proteins (such as Bcl-2) have been implicated in cancer progression. Introduction of an antisense molecule specific for the overexpressed genes or homologous recombination of an endogenous locus with a “normal” copy of the gene delivered using the complex of the invention can be therapeutically beneficial. In a similar fashion, delivery of entire organelles can also effect the same therapeutic results, particularly for conditions characterized by organelle dysfunction, such as those listed herein.

It is to be understood that in addition to coupling the complexes of the invention to the targeting sequences listed herein, the nucleic acid molecules and/or organelles to be delivered can be ligated to other molecules that direct localization within a cell. If nucleic acids are being delivered, then they may be conjugated to other nucleic acid sequences that encode for a targeting sequence. In this latter instance, the expressed peptide can be transported to a particular location (such as a particular organelle or cell). Organelles on the other hand may be more preferably conjugated to peptide sequences that effect their delivery to a desired location.

As used herein, an “organelle targeting compound” is a compound that directs the non-viral nucleic acid and/or organelle delivery complex to a particular organelle or set of organelles. For example, the organelle targeting compound can direct the non-viral nucleic acid and/or organelle delivery complex to the nucleus, mitochondria, chloroplast (provided the cell is a plant cell), Golgi apparatus, endoplasmic reticulum, and the like. Commonly, organelle targeting compounds are peptide in nature.

Compounds that effect transport into the nucleus are known, and these include the nuclear leader sequence of the simian virus SV40 large tumor antigen (KKKRK) (SEQ ID NO:5), importin binding nuclear targeting sequences, importin and karyoopherin localization sequences, and classical nuclear localization sequences (NLS) such as influenza virus nucleoprotein C-terminal residues 336-345 (AAFEDLRVLS) (SEQ ID NO:6), adenovirus E1a C-terminal amino acids (KRPRP) (SEQ ID NO:7), yeast mata2 residues 1-13 (KIPIK) (SEQ ID NO:8), and yeast ribosomal protein L3 residues 1-21 (PRKR) (SEQ ID NO:9). (Pap et al. Exp. Cell. Res. 265:288-293, 2001; Smith et al. Plant Cell 10:1791-1799, 1998; Jans et al. Bioessays 22:532-544, 2000; Dingwall et al. Ann. Rev. Cell. Biol. 2:367-390, 1986; Lyons et al. Mol. Cell. Biol. 7:2451-2456, 1987.) These compounds are referred to herein as nuclear targeting compounds.

Compounds that effect transport into the mitochondria are known, and these include the signal sequence of Cbs2p (25 N-terminal amino acids); Tom20- and Tom70-binding mitochondrial presequences; Cit1p mitochondrial targeting sequence (N-terminus), and cytochrome b2 sorting sequence. (Huang et al., FEBS Lett. 487:248-51, 2000; Tzschoppe et al. Biol Chem. 381:1175-1183, 2000; Muto et al. J. Mol. Biol. 306:137-143, 2001; Lee et al. J. Biochem. 128:1059-1072, 2000; Geissler et al. Mol. Biol. Cell. 11:3977-3991, 2000; Brix et al. J. Mol. Biol. 303:479-488, 2000.) In addition, most mitochondrial proteins (i.e., proteins found within the mitochondria) contain “uptake-targeting sequences” in their N-terminal 20-60 amino acid residues. Localization within the mitochondria is also possible by coupling the complex to matrix targeting sequences or intermembrane-space targeting sequences. These compounds are referred to herein as mitochondria targeting compounds such as BNIP3 with the BH3 domain deleted or BNIP1. (Yasuda et al., J Biol Chem 1998 273(20):12415-12421.)

Compounds that effect transport into a chloroplast are known, and these include the N-terminal transit sequence or transit peptide of the small subunit of ribulose-1,5-biosphosphate carboxylase/oxygenase (Rubisco). (Robinson et al. Traffic 2:245-251, 2001; Liu et al. Biochem. J. 351:377-84, 2000; Bruce Trends Cell. Biol. 10:440-447, 2000.) These compounds are referred to herein as chloroplast targeting compounds.

Compounds that effect transport into the endoplasmic reticulum (ER) are known, and these include the ER sequence from HSP47, amino acid sequences KDEL (SEQ ID NO: 10), the N-terminal ER-specific signal peptide from a Phaseolus vulgaris lectin gene, and signal peptides from proproalbumin (MKWVTFLLLLFISGSAFSR) (SEQ ID NO: 11), pre-IgG light chain (MDMRAPAQIFGFLLLLFPGTRCD) (SEQ ID NO:12), prelysozyme (MRSLLILVLCFLPLAALGK) (SEQ ID NO:13), preprolactin (MNSQVSARKAGTLLLLMMSNLL) (SEQ ID NO:14), prepenicillinase (E. coli) (MSIQHFRVALIPFFAFCLPVFAH) (SEQ ID NO:15), and prevesicular stomatitis virus (VSV) glycoprotein (KCLLYLAFLFIHVNCK) (SEQ ID NO:16), prelipoprotein (E. coli) (MKATKLVLGAVILGSTLLAGC) (SEQ ID NO:17). (Matsuda et al. Biochem. Biophys. Res. Commun. 280:53540, 2001; Pap et al. Exp. Cell. Res. 265:288-293, 2001; Sherritt et al. Int. limunol. 13:265-271, 2201; Boehm et al. Transgenic Res. 9:477-486, 2000; Leader TIBS 4:205, 1979.) These compounds are referred to herein as ER targeting compounds.

Compounds that effect transport into peroxisomes are known, and these include Cit2p peroxisomal targeting sequence (C-terminus), and PTS 1 sequence AKL. (Lee et al. J. Biochem. 128:1059-1072, 2000; Pap et al. Exp. Cell. Res. 265:288-293, 2001.) These compounds are referred to herein as peroxisomal targeting compounds.

Compounds that effect transport into trans-Golgi network are known, and these include the amino acid sequence SDQRL (SEQ ID NO:18). (Pap et al. Exp. Cell. Res. 265:288-293, 2001.) Yet other known systems for protein translocation include the twin-arginine translocation (Tat) system. (Robinson, Biol Chem. 381:89-93, 2000.) These compounds are referred to herein as Golgi targeting compounds.

As used herein, a “cytoskeleton targeting compound” is a compound that directs the non-viral nucleic acid and/or organelle delivery complex to the cytoskeleton of the cell, and preferably allows the non-viral nucleic acid and/or organelle delivery complex to move throughout the cell including, for example, to its intended destination in the cell. These compounds may bind cytoskeletal markers or they may bind to agents that are bound to the cytoskeleton. Examples of cytoskeleton targeting compounds include importin a, a protein (Smith et al. Plant Cell 10:1791-1799, 1998), tobacco mosaic virus movement protein (Heinlein et al., Science 270:1983-1985, 1995; McLean et al., Plant Cell 7:2101-2114), and elongation factor-1α (Durso et al., Protoplasma 180:99-105, 1994; Clore et al., Plant Cell 8:2003-2014, 1996.), all of which reportedly coalign with microtubules and microfilaments. Other examples include the amino terminal end of IκBα that has been shown to associate with D1c-1 (a dynein family member that associates with the cytoskeleton), D1c-1, (Crepieux et al., Mol. Cell. Biol. 17:7375-7385, 1997.)

The non-viral nucleic acid and/or organelle delivery complex may be administered directly to a tissue. Preferably, the tissue is a tissue in which a disorder exists, or in which a disorder is likely to exist. Direct tissue administration may be achieved by direct injection. The non-viral nucleic acid and/or organelle delivery complex may be administered once, or alternatively it may be administered in a plurality of administrations. If administered multiple times, the non-viral nucleic acid and/or organelle delivery complex may be administered via different routes. For example, the first (or the first few) administrations may be made directly into the affected tissue or joints while later administrations may be systemic. These later administrations may also comprise lower doses of non-viral nucleic acid delivery complex.

In an important embodiment, the non-viral nucleic acid and/or organelle delivery complex targeted to nervous system cells such as astrocytes, neurons, retinal ganglion cells, macrophages, microglia, and other immune cells. It has been reported that neurons contain high levels of GM1, the receptor for CTB. (Shogomori et al. J. Biol. Chem. 276:9182-9188, 2001.) Accordingly, a natural target for the complexes of the invention is neurons and their support cells.

Other particularly important sites for nucleic acid molecule and/or organelle delivery include the retina, the central nervous system including the spinal cord and peripheral nerves, the lungs, the muscles, the bone marrow, the breast, joints, kidneys, bladder, prostate, and the skin.

The non-viral delivery complex may also be targeted to a cell or tissue using a “cell targeting compound.” As used herein, a cell targeting compound is a compound that directs the non-viral delivery complex to a particular cell, tissue or organ. The complexes may be targeted to primary or, in some instances, secondary (i.e., metastatic) lesions through the use of cell targeting compounds which preferentially recognize a cell surface marker.

The cell targeting compound of choice will depend upon the nature of, for example, the disorder sought to be treated or prevented. Examples of compounds that localize to malignant breast epithelium include, but are not limited to, estrogen and progesterone, COX-2 among others. Ovarian cancers are also known to express EGFR and c-fms, and thus could be targeted through the use of ligands for either receptor. In the case of c-fms which is also expressed by macrophages and monocytes, targeted delivery to an ovarian cancer may require a combination of local administration such as a vaginal suppository as well as a targeting compound. Prostate cancers can be targeted using compounds such as peptides (e.g., antibodies or antibody fragments) which bind to prostate specific antigen (PSA) or prostate specific membrane antigen (PSMA). Other markers which may be used for targeting of the agent to specific tissues include, for example, in liver: HGF, insulin-like growth factor I, II, insulin, OV-6, HEA-125, hyaluronic acid, collagen, N-terminal propeptide of collagen type III, mannose/N-acetylglucosamine, asialoglycoprotein, tissue plasminogen activator, PAF-AH, low density lipoprotein, carcinoembryonic antigen; in kidney cells: angiotensin II, vasopressin, antibodies to CD44v6; in keratinocytes and skin fibroblasts: KGF, very low density lipoprotein, RGD-containing peptides, collagen, laminin; in melanocytes: kit ligand; in gut: cobalamin-intrinsic factor, heat stable enterotoxin of E. coli; in breast epithelium: heregulin, prolactin, transferrin, cadherin-11. Other markers specific to particular tissues are available and would be known to one of ordinary skill in the art. In still other embodiments, the complex of the invention may be targeted to fibroblasts via ligands or binding partners for fibroblast specific markers. Examples of these markers include, but are not limited to fibroblast growth factors (FGF) and platelet derived growth factor (PDGF).

The targeting compounds can be physically combined with the complex of the invention without bonding. The targeting compounds may also be directly conjugated via a covalent linkage or an ionic bond, or they may also be indirectly conjugated to the complexes via a linking molecule. Methods of conjugation suitable in the invention are described below.

It is to be understood that the invention embraces a variety of linkages known in the art for coupling CTB, cyclodextrin, and any of the targeting moieties described herein to each other, if desired. The components of the non-viral delivery complex may be coupled to each other via linldng molecules. Examples of suitable linking molecules which can be used include bifunctional crosslinker molecules. The bifunctional crosslinker molecules may be homobifunctional or heterobifunctional, depending upon the nature of the molecules to be conjugated. Homobifunctional crosslinkers have two identical reactive groups. Heterobifunctional crosslinkers are defined as having two different reactive groups that allow for sequential conjugation reaction. Various types of commercially available crosslinkers are reactive with one or more of the following groups: primary amines, secondary amines, sulphydryls, carboxyls, carbonyls and carbohydrates. Examples of amine-specific crosslinkers are bis(sulfosuccinimidyl) suberate, bis[2-(succinimidooxycarbonyloxy)ethyl]sulfone, disuccinimidyl suberate, disuccinimidyl tartarate, dimethyl adipimate.2 HCl, dimethyl pimelimidate.2 HCl, dimethyl suberimidate.2 HCl, and ethylene glycolbis-[succinimidyl-[succinate)]]. Crosslinkers reactive with sulfhydryl groups include bismaleimidohexane, 1,4-di-[3′-(2′-pyridyldithio)-propionamido)]butane, 1-[p-azidosalicylamido]-4-[iodoacetamido]butane, and N-[4-(p-azidosalicylamido) butyl]-3′-[2′-pyridyldithio]propionamide. Crosslinkers preferentially reactive with carbohydrates include azidobenzoyl hydrazine. Crosslinkers preferentially reactive with carboxyl groups include 4-[p-azidosalicylamido]butylamine. Heterobifunctional crosslinkers that react with amines and sulfhydryls include N-succinimidyl-3-[2-pyridyldithio]propionate, succinimidyl[4-iodoacetyl]aminobenzoate, succinimidyl 4-[N-maleimidomethyl) cyclohexane-1-carboxylate, m-maleimidobenzoyl-N-hydroxysuccinimide ester, sulfosuccinimidyl 6-[3-[2-pyridyldithio]propionamido]hexanoate, and sulfosuccinitnidyl 4-[N-maleimidomethyl]cyclohexane-1-carboxylate. Heterobifunctional crosslinkers that react with carboxyl and amine groups include 1-ethyl-3-[[3-dimethylaminopropyl] carbodiimide hydrochloride. Heterobifunctional crosslinkers that react with carbohydrates and sulfhydryls include 4-[-maleimidomethyl]-cyclohexane-1-carboxylhydrazide.2 HCl, 4-(4-N-maleimidophenyl)-butyric acid hydrazide.2 HCl, and 3-[2-pyridyldithio]propionyl hydrazide. The crosslinkers are bis-[β-4-azidosalicylamido)ethyl]disulfide and glutaraldehyde. Amine or thiol groups may be added at any nucleotide of a synthetic nucleic acid molecule so as to provide a point of attachment for a bifunctional crosslinker molecule. The nucleic acid molecule may be synthesized incorporating conjugation-competent reagents such as Uni-Link AminoModifier, 3′-DMT-C6-Amine-ON CPG, AminoModifier II, N-TFA-C6-AminoModifier, C6-ThiolModifier, C6-Disulfide Phosphoramidite and C6-Disulfide CPG (Clontech, Palo Alto, Calif.).

Alternatively, the targeting compound may be conjugated or associated with an intermediary compound such as, for example, a liposome within which the complex and/or nucleic acid molecule is encapsulated. Liposomes are artificial membrane vessels which are useful as a delivery vector in vivo or in vitro. It has been shown that large unilamellar vessels (LUV), which range in size from 0.2-4.0 μm can encapsulate large macromolecules. Liposomes may be targeted to a particular tissue, such as the vascular cell wall, by coupling the liposome to a specific ligand such as a monoclonal antibody, sugar, glycolipid, or protein. Liposomes are commercially available from Gibco BRL, for example, as LIPOFECTIN™ and LIPOFECTACE™, which are formed of cationic lipids such as N-[1-(2, 3 dioleyloxy)-propyl]-N,N,N-trimnethylammonium chloride (DOTMA) and dimethyl dioctadecylammonium bromide (DDAB). Methods for making liposomes are known in the art and have been described in many publications. Liposomes also have been reviewed by Gregoriadis, G. in Trends in Biotechnology, V. 3, p. 235-241 (1985). In still other embodiments, the targeting compound may be loosely associated with the complex, such as within a microparticle comprising a polymer, the complex and the targeting compound.

The invention intends to target disorders (and subjects having, or at risk of developing, such disorders) that are reported to have a genetic basis, via the delivery of nucleic acid molecules and/or organelles. In some instances, these disorders have a monogenic basis, meaning that the disorder arises from mutation at a single genetic locus (i.e., a single gene).

The methods can be equally applied to disorders having a polygenic basis (i.e., mutation of more than one genetic locus) as well. Treatment of polygenic disorders can be achieved via the delivery of nucleic acid molecules and/or organelles corresponding to single or multiple genetic loci. Disorders that can be targeted include but are not limited to neurodegenerative disorders (see below), muscular degenerative disorders such as muscular dystrophy and amyotrophic lateral sclerosis, cystic-fibrosis, immunodeficiencies such as severe combined immunodeficiency disease, autoimmune disorders such as arthritis, systemic lupus erythematosus, and multiple sclerosis, cancers such as colon cancer, retinoblastoma and metabolic disorders. The diagnosis of subjects having or at risk at developing such disorders is within the realm of a medical practitioner of ordinary skill.

Once delivered to the cell, the nucleic acid molecule can provide a variety of functions such as promoting or inhibiting the synthesis of additional nucleic acid molecules, such as RNA molecules or antisense molecules, or polypeptide upon cell entry. The end result of the nucleic acid molecule transfer can be to alter gene expression which in turn can influence cell growth, cell differentiation or cell death, depending on the nature and function of the encoded polypeptide(s) and the specific application. The nucleic acid molecule can encode one or more native polypeptides which may be absent or defective due to mutation in the wild type gene. For example, the nucleic acid molecule may comprise homologous sequences which facilitate its recombination with host cell sequences in order to repair damaged or mutant genes or to simply increase the number of copies of a normal gene.

In general, any peptide based therapeutic agent can be delivered (in nucleic acid form) to a target cell using the methods and compositions of the invention. A list of peptide based therapeutic agents is provided in published PCT Patent Application WO97/16169, published May 9, 1997.

In some important embodiments, the methods and compositions provided herein are used to delivery specific nucleic acid molecules and/or organelles to neural cells, particularly for the therapeutic benefit of neural degenerative disorders such as Alzheimer's disease, Parkinson's disease, Huntington's disease, Canavan's disease, Chediak-Higashi syndrome, Usher syndrome, MS, and AIDS related dementia. Many of these disorders have been identified as having a genetic basis. The genes involved in some of these disorders include huntingtin (Huntington's disease), parkin (autosomal recessive juvenile parkinsonism), alpha-synuclein, tau, UCH-L1 and parkin (familial parkinsonism), aspartoacylase (Canavan's disease), CHS1/beige (Chediak-Higashi syndrome), amyloid precursor protein, presenilin 1 (PS1), presenilin 2 (PS2), α2-macroglobulin, low density lipoprotein receptor related gene, and apolipoprotein E ε4 (Alzheimer's disease).

In one embodiment, the invention can be used to create dopaminergic cells in subjects having for example Parkinson's disease. As an example, epidermal cells or other undifferentiated stem cells can be used to create dopaminergic cells and these cells can be transplanted into a subject in the area of the substantia nigra, or basal ganglia in the forebrain. This can also be done to produce Ach cells that are then transplanted into the basal forebrain in the treatment of Alzheimer's disease.

In another embodiment, the invention can be used to increase the expression of A β regulatory protein in subjects having or at risk of developing Alzheimer's disease. This protein drives cleavage of amyloid precursor protein to a non P cleavage site, limiting the creation of the A β peptide.

In another embodiment, the invention provides for the transfection of nucleic acid molecules that encode growth factors (such as nerve growth factor (NGF)) and/or other mitogenic peptides and polypeptides into the brain in neurodegenerative diseases such as those listed above. Many of the cells that degenerate in these disorders are dependent upon NGF and other mitogenic peptides and polypeptides that are retrogradely transported to keep cells viable and healthy. For instance, basal forebrain cholinergic cell death is a hallmark of Alzheimer's disease. By increasing NGF levels, the amount of cell death can be reduced, the cortex and hippocampus remains innervated by basal forebrain neurons, and the associated symptoms can be alleviated.

In yet other embodiments, the method is used to transfect nerve cells with cytokines such as IL-4 or erythropoietin.

In another embodiment, nucleic acid molecules encoding IFκB are transfected into the brains of subjects having nervous system disorders resulting from, at least in part, an immune system abnormality. Examples of these disorders include asthma, related nervous system disorders, and Alzheimer's disease. A hallmark of immune system overactivity is an increase in the level of the transcription factor NFκB, which subsequently enhances the expression of other immunoregulators such as COX-2 and TNF alpha. IFκB binds to and thereby interferes with the transcriptional activity of NFκB. By increasing the levels of the IFκB, the activity of NFκB is reduced as is the level of integral immunoregulators downstream of NFκB.

IFκB delivery is also therapeutically beneficial in other inflammatory disorders such as arthritis, inflammatory bowel disease such as Crohn's disease, Sjogren syndrome, retroperitoneal inflammation; asthma; blepharitis; bronchitis; cervicitis; conjunctivitis; esophagitis; glossitis; myocarditis; proctitis; rashes; retroperitoneal fibrosis; scleritis; achilles tendonitis; acute appendicitis; acute bronchitis; acute cholecystitis; acute nephritic syndrome; acute pancreatitis; acute pulmonary eosinophilia (loeffler's syndrome); adhesive capsulitis; alcoholic liver disease (hepatitis/cirrhosis); allergic vasculitis; ankylosing spondylitis; arterial insufficiency; aseptic meningitis; atopic dermatitis; atrophic vaginitis; autoimmune hepatitis; bacterial gastroenteritis; balanitis; bronchiolitis; bunions; bursitis; caplan's syndrome; cellulitis; cerebral abscess; chemical pneumonitis; cholangitis; chronic active hepatitis; chronic bronchitis; chronic cholecystitis; chronic glomerulonephritis; chronic inflammatory polyneuropathy; chronic pancreatitis; chronic persistent hepatitis; chronic thyroiditis (hashimoto's disease); chronic urethritis; chylomicronemia syndrome; cmv gastroenteritis/colitis; colds; colitis; colloid nodular goiter; compartment syndrome; contact dermatitis; costochondritis; croup; culture-negative endocarditis; dacryoadenitis; dermatis herpetiformis; dermatomyositis; diskitis; diverticulitis; drug-induced hepatitis; E. coli enteritis; echo virus infection; encephalitis; encephalitis (meningoencephalitis); endocarditis; endometritis; endophthalmitis; enteritis; eosinophilic fasciitis; epididymitis; epidural abscess; epiglottitis; episcleritis; erysipelas; esophagitis herpes; ethmoiditis; familial mediterranean fever; feldene overdose; fenoprofen calcium overdose; flurbiprofen overdose; foreign object aspiration or ingestion; fungal arthritis; gallbladder disease; gastritis; gastritis-chronic; gastritis-acute; gastroesophageal reflux disease; giant cell (temporal, cranial) arteritis; gingivitis; glomerulonephritis; goiter; goodpasture's syndrome; guillain-barre; helicobacter pylori gastritis (chronic gastritis); hepatitis; hepatitis a; hepatitis b; hepatitis c; herpetic stomatitis; hospital-acquired pneumonia; hypersensitivity pneumonitis; iga nephropathy (berger's disease); igm mesangial proliferative glomerulonephritis; indocin overdose; industrial bronchitis; interstitial keratitis; interstitial nephritis; intertrigo; ischemic colitis; joint pain; labyrinthitis; laryngitis; lateral epicondylitis/tennis elbow; lichen planus; lung gallium (ga.) scan; lyme disease; lyme disease-primary; lymphogranuloma venereum; mediastinitis; meibomianitis; membranoproliferative gn i; membranoproliferative gn ii; membranous nephropathy; meningitis; meningitis; cryptococcal; meningitis; gram negative; meningitis; h. influenza; meningitis; meningococcal; meningitis; pneumococcal; meningitis; staphylococcal; myositis; neurosarcoidosis; non-gonococcal (septic) bacterial arthritis; optic neuritis; orchitis; otitis extema; acute; otitis externa; chronic; otitis externa; malignant; otitis media; acute; otitis media; chronic; painless (silent) thyroiditis; pancreatitis; pericarditis; pericarditis; bacterial; pericarditis; constrictive; pericarditis; post-mi; periodontitis; perioral dermatitis; perirenal abscess; peritonitis; peritonitis; dialysis associated; peritonitis; secondary; peritonitis; spontaneous; petrositis; pharyngitis; pharyngitis-viral; pharyngitis, gonococcal; pharyngitis, streptococcal; pharyngomaxillary space abscess; pityriasis rubra pilaris; pleurisy; pneumonia; pneumonia in immunocompromised host; pneumonia with lung abscess; polymyositis (adult); post-streptococcal gn; proctitis-streptococcal; progressive multifocal leukoencephalopathy; prostatitis; acute; prostatitis; chronic; prostatitis; non-bacterial; radiation enteritis; reiter's syndrome; retinopathy; retrocalcaneal bursitis; rosacea; rotator cuff tendonitis; sarcoidosis; sclerosing cholangitis; shigella enteritis; sinusitis; chronic; sleeping sickness; spinal cord abscess; stress gastritis; subacute thyroiditis; superficial thrombophlebitis; tendinitis; tenosynovitis; thromboangiitis obliterans; thrombophlebitis; tonsillitis; trigeminal neuralgia; tuberculous arthritis; urethritis; uveitis; uvulitis; vernal conjunctivitis; viral arthritis; viral gastroenteritis; viral pneumonia; vulvitis; vulvovaginitis; wbc (nuclear) scan; wegener's granulomatosis.

In still other embodiments, the methods and compositions of the invention can be used to deliver a “suicide” gene to a cell either in vivo or in vitro. As used herein, a “suicide” gene refers to a gene which either directly or indirectly leads to the death of the cell which expresses it. One example of a well-known suicide gene is the herpes simplex virus thymidine kinase (HS-TK) gene, which has been used to kill brain tumor cells in vivo. Expression of the HS-TK gene renders a cell sensitive to the antiviral drugs gancyclovir and acyclovir. Another suicide gene is bacterial enzyme cytosine dearninase that converts exogenously administered 5-fluorocytosine to 5-fluorouracil (5-FU). 5FU in turn induces its cytotoxic effects through its active metabolite 5-fluorodeoxyuridine monophosphate (fdUMP) which irreversibly binds to and inhibits the action of thymidylate synthetase, the enzyme responsible for converting deoxyuridine monophosphate (dUMP) to thymidine monophosphate.

Suicide gene therapy is best suited where it is desirable to remove or eliminate cells from a subject or in vitro. Accordingly, cancer cells are suitable targets for suicide gene therapy. Of particular interest are cancers of the brain such as astrocytoma, craniopharyngioma, ependymoma, glioma (e.g., brain stem glioma), medulloblastoma, neuroblastomas and retinoblastoma. The invention can also be used to treat other types of cancers, including cancers that metastasize to the brain (i.e., metastatic brain tumors), biliary tract cancer; breast cancer; cervical cancer; choriocarcinoma; colon cancer; endometrial cancer; esophageal cancer; gastric cancer; hematological neoplasms, including acute lymphocytic and myelogenous leukemia; chronic lymphocytic and myelogenous leukemia; multiple myeloma; AIDS-associated leukemias and adult T-cell leukemia lymphoma; intraepithelial neoplasms, including Bowen's disease and Paget's disease; liver cancer; lung cancer; lymphomas, including Hodgkin's disease and lymphocytic lymphomas; oral cancer, including squamous cell carcinoma; ovarian cancer, including those arising from epithelial cells, stromal cells, germ cells, and mesenchymal cells; pancreas cancer; prostate cancer; colorectal cancer; sarcomas, including leiomyosarcoma, rhabdomyosarcoma, liposarcoma, fibrosarcoma, and osteosarcoma; skin cancer, including melanoma, Kaposi's sarcoma, basocellular cancer, and squamous cell cancer; testicular cancer, including germinal tumors (seminoma, non-seminoma teratomas, and choriocarcinomas), stromal tumors, and germ cell tumors; thyroid cancer, including thyroid adenocarcinoma and medullar carcinoma; and renal cancer including adenocarcinoma and Wilms' tumor.

The invention provides for the delivery of nucleic acid molecules that encode tumor suppressor genes, particularly in the treatment of cancer. Tumor suppressor genes are commonly inactivated in cancerous lesions. Examples of tumor suppressor genes include but are not limited to p53, Rb and WT-1.

Other genetic mutations which can be targeted are known in the art and include CFTR (cystic fibrosis), and dystrophin (muscular dystrophy) mutations. In another embodiment, the nucleic acid molecule may encode a polypeptide which acts to inactivate dominant negative proteins which result from mutation.

The invention can also be used to pre-condition cells prior to transplant or re-introduction into a subject, or to modulate or influence cell migration upon transplant.

As used herein, a nucleic acid molecule refers to DNA, RNA and DNA/RNA hybrid molecules composed of native and/or modified nucleotides. In some embodiments of the invention, the nucleic acid molecule is a sense DNA or RNA molecule, in a single or double stranded form depending on the intended application of the invention. In other embodiments, the nucleic acid molecule is an antisense DNA or RNA molecule. As used herein, the term “transduced nucleic acid molecule” is used interchangeably with delivered nucleic acid molecule, transferred nucleic acid molecule and nucleic acid molecule to be delivered. Similarly the term transduction is meant to be used interchangeably with delivery, transfection and transfer.

As used herein with respect to nucleic acid molecules, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid molecule is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid molecule may be substantially purified, but need not be. For example, a nucleic acid molecule that is isolated within a cloning or expression vector is not pure in that it may comprise only a tiny percentage of the material in the cell in which it resides. Such a nucleic acid molecule is isolated, however, as the term is used herein because it is readily manipulable by standard techniques known to those of ordinary skill in the art.

As used herein with respect to peptides as well as non-peptide based compounds, the term “isolated” means separated from its native environment in sufficiently pure form so that it can be manipulated or used for any one of the purposes of the invention. Thus, isolated means sufficiently pure to be used (i) to raise and/or isolate antibodies, (ii) as a reagent in an assay, or (iii) for sequencing, etc.

The nucleic acid molecules to be delivered are generally present in a vector, particularly if they are intended to express a peptide or polypeptide. As used herein, a “vector” may be any of a number of nucleic acid molecules into which a desired sequence may be inserted by restriction and ligation for transport between different genetic environments or for expression in a host cell. Vectors are typically composed of DNA although RNA vectors are also available. Vectors include, but are not limited to, plasmids, phagemids and virus genomes. A cloning vector is one which is able to replicate in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host bacterium or just a single time per host before the host reproduces by mitosis. In the case of phage, replication may occur actively during a lytic phase or passively during a lysogenic phase.

An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase or alkaline phosphatase), and genes which visibly affect the phenotype of transformed or transfected cells, hosts, colonies or plaques. Preferred vectors are those capable of autonomous replication and expression of the structural gene products present in the DNA segments to which they are operably joined.

The expression vectors of the present invention include regulatory sequences operably joined to a nucleotide sequence encoding a peptide or polypeptide. As used herein, the term “regulatory sequences” means nucleotide sequences which are necessary for or conducive to the transcription of a nucleotide sequence which encodes a desired peptide and/or which are necessary for or conducive to the translation of the resulting transcript into the desired peptide. Regulatory sequences include, but are not limited to, 5′ sequences such as operators, promoters and ribosome binding sequences, and 3′ sequences such as polyadenylation signals. The vectors of the invention may optionally include 5′ leader or signal sequences, 5′ or 3′ sequences encoding fusion products to aid in protein purification, and various markers which aid in the identification or selection of transformants. The choice and design of an appropriate vector is within the ability and discretion of one of ordinary skill in the art. The subsequent purification of the peptides or polypeptides may be accomplished by any of a variety of standard means known in the art.

Tissue specific promoters that are known in the art and which can be readily used in the nucleic acid molecules of the invention include prion protein promoter for brain specific expression, neuron-specific enolase promoter for brain specific expression, CaMKII-alpha promoter for forebrain specific expression, LAP promoter for liver specific expression, 5′ flanking sequence from the rat alpha-myosin heavy chain gene for heart specific expression, Clara cell 10 kD protein (CC 10) promoter for lung specific expression, mouse mammary tumor virus long terminal repeat for salivary gland specific expression, human keratin 14 (K14) promoter for epithelial specific expression, bovine keratin 6 (K6) promoter/regulatory region for hair follicle keratinocyte specific expression, Tek or Tie promoter for endothelial specific expression, mouse whey acidic protein (WAP) promoter for mammary gland specific expression, β-lactoglobulin promoter for mammary gland specific expression, albumin promoter for liver specific expression, myosin light chain I promoter for muscle specific expression, endothelial specific promoters, tyrosine kinase promoters for melanoma specific expression, DF3/MUC1 promoter for breast cancer specific expression, and afetoprotein promoter for hepatoma specific expression.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. Especially, such 5′ non-transcribed regulatory sequences will include a promoter region which includes a promoter sequence for transcriptional control of the operably joined gene. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989.

Preferred systems for mRNA expression in mammalian cells are those such as pcDNA3.1 (available from Invitrogen, Carlsbad, Calif.) that contain a selectable marker such as a gene that confers G418 resistance (which facilitates the selection of stably transfected cell lines) and the human cytomegalovirus (CMV) enhancer-promoter sequences. Additionally, suitable for expression in primate or canine cell lines is the pCEP4 vector (Invitrogen, Carlsbad, Calif.), which contains an Epstein Barr virus (EBV) origin of replication, facilitating the maintenance of plasmid as a multicopy extrachromosomal element. Another expression vector is the pEF-BOS plasmid containing the promoter of polypeptide Elongation Factor 1α, which stimulates efficiently transcription in vitro. The plasmid is described by Mishizuma and Nagata (Nuc. Acids Res. 18:5322, 1990), and its use in transfection experiments is disclosed by, for example, Demoulin (Mol. Cell. Biol. 16:4710-4716, 1996). Still another preferred expression vector is an adenovirus, described by Stratford-Perricaudet, which is defective for E1 and E3 proteins (J. Clin. Invest. 90:626-630, 1992). The use of the adenovirus as an Adeno.PlA recombinant is disclosed by Warnier et al., in intradermal injection in mice for immunization against P1A (Int. J. Cancer, 67:303-310, 1996). Other examples of mammalian expression vectors include pCDM8 (Seed, B. (1987) Nature 329:840) and pMT2PC (Kaufman et al. (1987) EMBO J. 6:187-195).

Examples of suitable inducible E. coli expression vectors include pTrc (Amann et al., (1988) Gene 69:301-315) and pET 11d (Studier et al., Gene Expression Technology: Methods in Enzvmology 185, Academic Press, San Diego, Calif. (1990) 60-89). Target gene expression from the pTrc vector relies on host RNA polymerase transcription from a hybrid trc-lac fusion promoter. Target gene expression from the pET 11d vector relies on transcription from a T7 gn10-lac fusion promoter mediated by a coexpressed viral RNA polymerase (17 gn1). This viral polymerase is supplied by host strains BL21 (DE3) or HMS174(DE3) from a resident λ prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid molecule to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

Examples of vectors for expression in yeast S. cerevisiae include pYepSec1 (Baldari, et al., (1987) EMBO 6:229-234), pMFa (Durjan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.).

Alternatively, vectors can be expressed in insect cells using baculovirus expression vectors. Baculovirus vectors available for expression of protein in cultured insect cells (e.g., Sf9 cells) include the pAc series (Smith et al. (1983) Mol. Cell Biol. 3:2156-2165) and the pVL series (Lucklow and Summers (1989) Virology 170:31-39).

Virtually any cells, prokaryotic or eukaryotic, which can be transformed with heterologous DNA or RNA and which optionally can be grown or maintained in culture, may be used in the practice of the invention. Examples include bacterial cells such as E. coli, insect cells (using baculovirus expression vectors), yeast cells, mammalian cells (e.g., mouse, hamster, pig, goat, primate, etc.), plant cells, insect cells, and the like. They may be of a wide variety of tissue types, and they may be primary cells or cell lines. Specific examples include CHO cells and COS cells. Cell-free transcription systems also may be used in lieu of cells. Suitable host cells are discussed further in Goeddel, Gene Expression Technology: Methods in Enzymology 185, Academic Press, San Diego, Calif. (1990). Alternatively, the expression vector can be transcribed and translated in vitro, for example using T7 promoter regulatory sequences and T7 polymerase.

The invention also embraces the use of the non-viral nucleic acid delivery complex to deliver antisense nucleic acid molecules. As used herein, the term “antisense oligonucleotide” or “antisense” describes an oligonucleotide that is an oligoribonucleotide, oligodeoxyribonucleotide, modified oligoribonucleotide, or modified oligodeoxyribonucleotide which hybridizes under physiological conditions to DNA comprising a particular gene or to an mRNA transcript of that gene and, thereby, inhibits the transcription of that gene and/or the translation of that mRNA.

The antisense molecules are designed so as to interfere with transcription or translation of a target gene upon hybridization with the target gene or transcript. Those skilled in the art will recognize that the exact length of the antisense oligonucleotide and its degree of complementarity with its target will depend upon the specific target selected, including the sequence of the target and the particular bases that comprise that sequence. It is preferred that the antisense oligonucleotide be constructed and arranged so as to bind selectively with the target under physiological conditions, i.e., to hybridize substantially more to the target sequence than to any other sequence in the target cell under physiological conditions.

Based upon allelic or homologous genomic and/or cDNA sequences, one of skill in the art can easily choose and synthesize any of a number of appropriate antisense molecules for use in accordance with the present invention. In order to be sufficiently selective and potent for inhibition, such antisense oligonucleotides should comprise at least 10 and, more preferably, at least 15 consecutive bases which are complementary to the target, although in certain cases modified oligonucleotides as short as 7 bases in length have been used successfully as antisense oligonucleotides (Wagner et al., Nat. Med 1(11): 1116-1118, 1995). Most preferably, the antisense oligonucleotides comprise a complementary sequence of 20-30 bases.

Although oligonucleotides may be chosen which are antisense to any region of the gene or mRNA transcripts, in preferred embodiments the antisense oligonucleotides correspond to N-terminal or 5′ upstream sites such as translation initiation, transcription initiation or promoter sites. In addition, 3′-untranslated regions may be targeted by antisense oligonucleotides. Targeting to mRNA splicing sites has also been used in the art but may be less preferred if alternative mRNA splicing occurs. In addition, the antisense is targeted, preferably, to sites in which mRNA secondary structure is not expected (see, e.g., Sainio et al., Cell Mol. Neurobiol. 14(5):439-457, 1994) and at which proteins are not expected to bind. One of ordinary skill in the art may easily derive the genomic DNA corresponding to a known mRNA or cDNA sequence. Thus, the present invention also provides for the delivery of antisense molecules to allelic, homologous and genomic DNAs.

In one set of embodiments, the antisense oligonucleotides of the invention may be composed of “natural” deoxyribonucleotides, ribonucleotides, or any combination thereof. That is, the 5′ end of one native nucleotide and the 3′ end of another native nucleotide may be covalently linked, as in natural systems, via a phosphodiester intemucleoside linkage. These oligonucleotides may be prepared by art recognized methods which may be carried out manually or by an automated synthesizer. They also may be produced recombinantly by vectors.

In preferred embodiments, however, the antisense oligonucleotides of the invention also may include “modified” oligonucleotides. That is, the oligonucleotides may be modified in a number of ways which do not prevent them from hybridizing to their target but which enhance their stability or targeting or which otherwise enhance their therapeutic effectiveness. The term “modified oligonucleotide” as used herein describes an oligonucleotide in which (1) at least two of its nucleotides are covalently linked via a synthetic internucleoside linkage (i.e., a linkage other than a phosphodiester linkage between the 5′ end of one nucleotide and the 3′ end of another nucleotide) and/or (2) a chemical group not normally associated with nucleic acid molecules has been covalently attached to the oligonucleotide. Preferred synthetic internucleoside linkages are phosphorothioates, alkylphosphonates, phosphorodithioates, phosphate esters, alkylphosphonothioates, phosphoramidates, carbamates, carbonates, phosphate triesters, acetamidates, carboxymethyl esters and peptides.

The term “modified oligonucleotide” also encompasses oligonucleotides with a covalently modified base and/or sugar. For example, modified oligonucleotides include oligonucleotides having backbone sugars which are covalently attached to low molecular weight organic groups other than a hydroxyl group at the 3′ position and other than a phosphate group at the 5′ position. Thus modified oligonucleotides may include a 2′ —O— alkylated ribose group. In addition, modified oligonucleotides may include sugars such as arabinose instead of ribose.

The non-viral nucleic acid delivery complex recognizes and binds nucleic acid molecules in a sequence independent manner. Accordingly, there is no restriction upon the nature of the nucleic acid molecules that can be delivered using these complexes.

The non-viral nucleic acid delivery complex and its method of use are amenable to nucleic acid molecule transduction in both in vitro and in vivo settings. When used in vitro, cells in suspension, or tissue or organ cultures can be used as targets for nucleic acid molecule delivery. When used in vivo, the nucleic acid molecules and/or organelles can be targeted to cells, tissue or organs as necessary. The method inherently avoids the risk of viral infection within a subject because it does not require viral proteins, capsids or the generation of recombinant viruses.

The methods of the invention are also useful for the ex vivo gene transfer into cells.

An example of an ex vivo gene therapy method is outlined in U.S. Pat. No. 5,399,346 and in exhibits submitted in the file history of that patent, all of which are publicly available documents. In general, ex vivo nucleic acid molecule delivery involves introduction in vitro of a functional copy of a gene into a cell(s) of a subject which contains a defective or absent copy of the wild type gene, and returning the genetically engineered cell(s) to the subject. In some embodiments, the functional copy of the gene is under operable control of regulatory elements which permit expression of the gene in the genetically engineered cell(s). In still other embodiments, the gene is under the control of native regulatory elements as described below.

The methods of the invention can be used for genetic screening based on phenotype or complementation assay following the introduction of large fragments of nucleic acid molecule into a cell or organism.

In other aspects, the complex can be used to screen for the function of select peptides that are conjugated to the complex. This could facilitate a screen for peptide sequences that permit introduction of selected reporter genes into specific tissues, cells, or intracellular locations. This method could be further adapted for introducing reporter genes to detect specific cellular defects associated with cancer or other diseases, or for introducing suicidal genes into diseased cells.

When the non-viral delivery complex is administered to a subject, the mode of administration and dosage of the complex will vary with the particular stage of the condition being treated, the age and physical condition of the subject being treated, the duration of the treatment, the nature of the concurrent therapy (if any), the specific route of administration, and the like factors within the knowledge and expertise of the health practitioner.

As used herein, a subject is a human, non-human primate, cow, horse, pig, sheep, goat, dog, cat or rodent. The methods of the invention can also be used to deliver nucleic acid molecules and/or organelles into plant cells.

When administered to a subject, the non-viral delivery complex of the invention is administered in a pharmaceutical preparation (or a pharmaceutical composition) commonly in combination with a pharmaceutically acceptable carrier. The term “pharmaceutically-acceptable carrier” as used herein means one or more compatible solid or liquid fillers, diluents or encapsulating substances which are suitable for administration into a human. The term “carrier” denotes an organic or inorganic ingredient, natural or synthetic, with which the non-viral nucleic acid delivery complex of the invention is combined to facilitate the application. The components of the pharmaceutical compositions also are capable of being co-mingled with the molecules of the present invention, and with each other, in a manner such that there is no interaction which would substantially impair the desired pharmaceutical efficacy.

Pharmaceutical preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

The pharmaceutical preparations, as described above, are administered in effective amounts. The effective amount will depend, as discussed above, upon the mode of administration, the particular condition being treated and the desired outcome. For therapeutic applications, if the nucleic acid molecule being delivered encodes a polypeptide, the effective amount may be that amount sufficient to cause expression of the nucleic acid molecule being delivered at a level to achieve a medically desirable result. If the nucleic acid molecule being delivered is an antisense nucleic acid molecule, the effective amount may be that amount to reduce the level of translation of a particular polypeptide in the targeted cell.

It may be possible in some embodiments to determine the effect of the transfected nucleic acid molecule or organelle by measuring cell proliferation, tumor size, or other physiological parameter. It is to be understood that in some embodiments the effective amount of the complex is not one which relies on the adjuvant activity of CTB. The amount of CTB administered can be an amount that does not stimulate an immune response when administered alone. This is the case particularly in embodiments that involve the delivery of nucleic acid molecules that do not encode a peptide or polypeptide, such as for example an antisense nucleic acid molecule.

Generally, doses of active compounds of the present invention would be from about 0.01 mg/kg per day to 1000 mg/kg per day. It is expected that doses ranging from 50-500 mg/kg will be suitable.

The invention also provides kits comprising the foregoing pharmaceutical preparation, and optionally instructions for use. A nucleic acid molecule and/or an organelle may also be provided in the kit.

The invention further provides a composition of the non-viral delivery complex for use as a medicament, methods for preparing the medicament, and methods for the sustained release of the medicament in vivo.

The pharmaceutical preparations, medicaments and kits of the invention can further comprise a therapeutic agent. A therapeutic agent is an agent that has been recognized as provided therapeutic or in some instanced prophylactic benefit to subjects having or at risk of developing a particular disorder. Examples of therapeutic agents include adrenergic agent; adrenocortical steroid; adrenocortical suppressant; alcohol deterrent; aldosterone antagonist; amino acid; ammonia detoxicant; anabolic; analeptic; analgesic; androgen; anesthesia, adjunct to; anesthetic; anorectic; antagonist; anterior pituitary suppressant; anthelmintic; anti-acne agent; anti-adrenergic; anti-allergic; anti-amebic; anti-androgen; anti-anemic; anti-anginal; anti-anxiety; anti-arthritic; anti-asthmatic; anti-atherosclerotic; antibacterial; anticholelithic; anticholelithogenic; anticholinergic; anticoagulant; anticoccidal; anticonvulsant; antidepressant; antidiabetic; antidiarrheal; antidiuretic; antidote; anti-emetic; anti-epileptic; anti-estrogen; antifibrinolytic; antifungal; antiglaucoma agent; antihemophilic; antihemorrhagic; antihistamine; antihyperlipidemia; antihyperlipoproteinemic; antihypertensive; antihypotensive; anti-infective; anti-infective, topical; anti-inflammatory; antikeratinizing agent; antimalarial; antimicrobial; antimigraine; antimitotic; antimycotic, antinauseant, antineoplastic, antineutropenic, antiobessional agent; antiparasitic; antiparkinsonian; antiperistaltic, antipneumocystic; antiproliferative; antiprostatic hypertrophy; antiprotozoal; antipruritic; antipsychotic; antirheumatic; antischistosomal; antiseborrheic; antisecretory; antispasmodic; antithrombotic; antitussive; anti-ulcerative; anti-urolithic; antiviral; appetite suppressant; benign prostatic hyperplasia therapy agent; blood glucose regulator; bone resorption inhibitor; bronchodilator; carbonic anhydrase inhibitor; cardiac depressant; cardioprotectant; cardiotonic; cardiovascular agent; choleretic; cholinergic; cholinergic agonist; cholinesterase deactivator; coccidiostat; cognition adjuvant; cognition enhancer; depressant; diagnostic aid; diuretic; dopaminergic agent; ectoparasiticide; emetic; enzyme inhibitor; estrogen; fibrinolytic; fluorescent agent; free oxygen radical scavenger; gastrointestinal motility effector; glucocorticoid; gonad-stimulating principle; hair growth stimulant; hemostatic; histamine H2 receptor antagonists; hormone; hypocholesterolemic; hypoglycemic; hypolipidemic; hypotensive; imaging agent; immunizing agent; immunomodulator; immunoregulator; immunostimulant; immunosuppressant; impotence therapy adjunct; inhibitor; keratolytic; LNRH agonist; liver disorder treatment; luteolysin; memory adjuvant; mental performance enhancer; mood regulator; mucolytic; mucosal protective agent; mydriatic; nasal decongestant; neuromuscular blocking agent; neuroprotective; NMDA antagonist; non-hormonal sterol derivative; oxytocic; plasminogen activator; platelet activating factor antagonist; platelet aggregation inhibitor; post-stroke and post-head trauma treatment; potentiator; progestin; prostaglandin; prostate growth inhibitor; prothyrotropin; psychotropic; pulmonary surface; radioactive agent; regulator; relaxant; repartitioning agent; scabicide; sclerosing agent; sedative; sedative-hypnotic; selective adenosine Al antagonist; serotonin antagonist; serotonin inhibitor; serotonin receptor antagonist; steroid; stimulant; suppressant; symptomatic multiple sclerosis; synergist; thyroid hormone; thyroid inhibitor; thyromimetic; tranquilizer; treatment of amyotrophic lateral sclerosis; treatment of cerebral ischemia; treatment of Paget's disease; treatment of unstable angina; uricosuric; vasoconstrictor; vasodilator; vulnerary; wound healing agent; xanthine oxidase inhibitor.

A variety of administration routes are available. The methods of the invention, generally speaking, may be practiced using any mode of administration that is medically acceptable, meaning any mode that produces effective levels of the active compounds without causing clinically unacceptable adverse effects. Such modes of administration include oral, rectal, topical, nasal, interdermal, transdermal, corneal, ocular, intraocular, intestinal, or parenteral routes. The term “parenteral” includes subcutaneous, intravenous, intramuscular, intrathecal or infusion. Intravenous or intramuscular routes are commonly used for gene delivery. Inhalation, e.g., by pulmonary aerosol, is also used for delivery of genes to cystic fibrosis patients. Intrathecal administration may be preferable where expression in nervous system cells is desired. Techniques for preparing aerosol delivery systems containing peptides are well known to those of skill in the art. Generally, such systems should utilize components which will not significantly impair the biological properties of the non-viral nucleic acid delivery complex (see, for example, Sciarra and Cutie, “Aerosols,” in Remington's Pharmaceutical Sciences, 18th edition, 1990, pp 1694-1712; incorporated by reference). Those of skill in the art can readily determine the various parameters and conditions for producing aerosols without resort to undue experimentation.

It has been reported that cyclodextrin readily permeates the skin. Nucleic acid molecules and/or organelles can be administered to the skin in order to treat skin related disorders. Alternatively, the delivery complexes may be intended for dendritic cells (particularly if the delivery complex delivers nucleic acid molecules) of the skin such as Langerhans cells, which can take up administered non-viral nucleic acid delivery complex, express and process the encoded antigen and then present it to other immune cells in the body of the subject in order to stimulate an antigen specific immune response. Such an approach is useful in the prevention and treatment of diseases that would benefit from an antigen specific immune response and these include but are not limited to infectious diseases such as bacterial infections, viral infections, fungal infections and parasitic infections; asthma and allergy, and cancer.

Cyclodextrins have also been reported to intravasate into the vasculature readily. Accordingly, cyclodextrin containing complexes can be used to deliver nucleic acid molecules or organelles systemically (via the bloodstream) in a subject.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Lower doses will result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of the nucleic acid molecule if systemic delivery is desirable.

Compositions suitable for oral administration may be presented as discrete units, such as capsules, tablets, lozenges, each containing a predetermined amount of the non-viral nucleic acid delivery complex of the invention. Other compositions include suspensions in aqueous liquids or non-aqueous liquids such as a syrup, elixir or an emulsion.

In yet other embodiments, the non-viral delivery complex is administered via a biocompatible microparticle or implant that is suitable for implantation into the mammalian recipient. Exemplary bioerodible implants that are useful in accordance with this method are described in PCT International Application No. PCT/US/03307 (Publication No. WO 95/24929, entitled “Polymeric Gene Delivery System”). PCT/US/0307 describes a biocompatible, preferably biodegradable polymeric matrix for containing a biological macromolecule. The polymeric matrix may be used to achieve sustained release of the agent in a subject. In accordance with one aspect of the instant invention, the complex described herein may be encapsulated or dispersed within the biocompatible, preferably biodegradable polymeric matrix disclosed in PCT/US/03307.

The polymeric matrix preferably is in the form of a microparticle such as a microsphere (wherein the agent is dispersed throughout a solid polymeric matrix) or a microcapsule (wherein the agent is stored in the core of a polymeric shell). Other forms of the polymeric matrix for containing the agent include films, coatings, gels, implants, and stents. The size and composition of the polymeric matrix device is selected to result in favorable release kinetics in the tissue into which the matrix device is implanted. The size of the polymeric matrix device further is selected according to the method of delivery which is to be used, typically injection into a tissue or administration of a suspension by aerosol into the nasal and/or pulmonary areas. The polymeric matrix composition can be selected to have both favorable degradation rates and also to be formed of a material which is bioadhesive, to further increase the effectiveness of transfer when the device is administered to a vascular, pulmonary, or other surface. The matrix composition also can be selected not to degrade, but rather, to release by diffusion over an extended period of time.

Both non-biodegradable and biodegradable polymeric matrices can be used to deliver the agents of the invention to the subject. Biodegradable matrices are preferred. Such polymers may be natural or synthetic polymers. Synthetic polymers are preferred. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multivalent ions or other polymers.

In general, the agents of the invention may be delivered using the bioerodible implant by way of diffusion, or more preferably, by degradation of the polymeric matrix. Exemplary synthetic polymers which can be used to form the biodegradable delivery system include: polyamides, polycarbonates, polyalkylenes, polyalkylene glycols, polyalkylene oxides, polyallkylene terepthalates, polyvinyl alcohols, polyvinyl ethers, polyvinyl esters, poly-vinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene and polyvinylpyrrolidone.

Examples of non-biodegradable polymers include ethylene vinyl acetate, poly(meth)acrylic acid, polyamides, copolymers and mixtures thereof.

Examples of biodegradable polymers include synthetic polymers such as polymers of lactic acid and glycolic acid, polyanhydrides, poly(ortho)esters, polyurethanes, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

Bioadhesive polymers of particular interest include bioerodible hydrogels described by H. S. Sawhney, C. P. Pathak and J. A. Hubell in Macromolecules, 1993, 26, 581-587, the teachings of which are incorporated herein, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Other delivery systems can include time-release, delayed release or sustained release delivery systems. Such systems can avoid repeated administrations, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-, di-, and tri-glycerides; hydrogel release systems; silastic systems; peptide based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775, 4,675,189, and 5,736,152 and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

Use of a long-term sustained release implant may be particularly suitable for treatment of chronic conditions. Long-term release, are used herein, means that the implant is constructed and arranged to deliver therapeutic levels of the non-viral delivery complex of the invention for at least 30 days, and preferably 60 days. Long-term sustained release implants are well-known to those of ordinary skill in the art and include some of the release systems described above.

The following examples are provided to illustrate specific instances of the practice of the present invention and are not to be construed as limiting the present invention to these examples. As will be apparent to one of ordinary skill in the art, the present invention will find application in a variety of compositions and methods.

### EXAMPLES

Introduction:

The transfection of the Bcl-2 gene and resulting production of Bcl-2 protein in the central nervous system (CNS) of adult hamsters has been accomplished with the mixing of two non-viral compounds. The experiment used adult hamsters, approximately 9 months old. The genetic material was encapsulated, and then linked to a targeting sequence to facilitate the entry of the genetic material into the cell. The ability to transfer nucleic acid molecules and/or organelles into cells has vast experimental and therapeutic implications. The transfection of Bcl-2, in particular has a dual purpose. The first is for its anti-apoptotic effect in a variety of tissues, including the CNS. The second is its growth promoting effect in a variety of tissues, such as for example retinal ganglion cells, after injury. This growth promoting activity may also be exploited in non-injured tissues according to the invention. The transfection of Bcl-2 via a non-viral in vivo method with resulting Bcl-2 protein production above 10% has been accomplished in the CNS of adult hamsters via punctate injections in the caudal superior colliculus (SC) as described herein. Further testing of this non-viral transfection method in retinal explant co-cultures has shown the method to be effective at a high rate of transfection through the production of enhanced green fluorescent protein (EGFP). The vehicles used to deliver the genetic material (and organelles, as the case may be) are combinations of Cholera toxin B subunit (CTB) complexed with a plasmid. The vehicles optionally contain a cyclodextrin such as β-cyclodextrin or γ-cyclodextrin. The complex of plasmid-CTB- γ-cyclodextrin was found to be the most effective, in some instances.

Other labs have focused on the problem of increasing the survival of neurons that are undergoing retrograde atrophic changes after axon transection (see, for example, Goldberg and Barres, 2000; Fournier, 1997; Di Polo, 1998; Sawai, 1996; Mansour-Robaey, 1994; Cohen, 1994; Thanos, 1997; Thanos and Mey, 1995; Agarwala and Kalil, 1998, Heiduschka and Thanos, 2000; Cho, et al., 1999; Cui, et al., 1999; Yip and So, 2000; Villegas-Perez, et al., 1988. This work has shown that application of trophic factors can enhance survival. Brain derived neurotrophic factor (BDNF) is required for the survival of developing neurons. (Altschuler, et al., 1999; Herzog and von Bartheld, 1998; Tonra, et al., 1998; Von Bartheld and Johnson, 2001). Also BDNF in culture has resulted in a reduction in oxidative stress. (Yamagata, et al., 1999). The age of the tissue appears to be important because if NGF is used on young tissue apoptosis will be triggered. Frade, et al., 1996; Kahle, et al., 1994; Khursigara, et al., 1999; Liepinsh, et al., 1997; MacPhee and Barker, 1997; Wheeler, et al.1998.

In addition, the secondary degenerative changes that cause lesions to enlarge after the initial trauma can be considerably reduced by this treatment. (Sabel and Schneider, 1988). When a neuron cannot take up a sufficient amount of trophic factors from its axonal endings, cell death may be triggered by apoptosis (Agarwala and Kalil, 1998; Aguayo, et al., 1996; Middleton, et al., 2000; Verney, et al., 2000; Williams, et al., 1986). Apoptosis is prevented during axonal elongation by sufficiently high levels of intracellular factors. Bcl-2 is one such factor. It becomes down regulated after the axon has reached its terminal area and begins to arborize for mouse RGCs. (See Chen, et al., 1997a). Presence of a Bcl-2 transgene with a promoter that allows transcription later in development can prevent the apoptosis consequent to axon transection (see, e.g., Chen, et al., 1997a). Therefore, transfection methods that insert Bcl-2 genes with such anomalous promoters into large numbers of neurons can be useful in inducing cell survival. The expression of Bcl-2 in primates has also been implicated in plasticity in the sub ventricular zone (SVZ). (Vinet, et al., 2002). Others have induced overexpression of Bcl-2 using adeno-associated virus (AAV) vectors and showed that Bcl-2 is able to protect the neurons in the lateral geniculate nucleus after visual cortex ablation in adult rats. (Caleo, et al., 2002). Some cell death by apoptosis may persist, however, because of other intracellular pathways (Chen, et al., 2001; Goswami and Dawson, 2000; Toman, et al., 2000). A recent study has shown that an anti-apoptotic drug, aurintricarboxylic acid, can increase retinal ganglion cell survival after optic nerve transection, and can more than double the number of axons regenerating into a peripheral nerve graft (Heiduschka and Thanos, 2000). Several other small molecules have been shown to enhance neuronal survival. LiCl is one such example that has been reportedly used inter alia in the retina. (Manji, et al., 2000; Zager and Ames, 1988). Additional work has been done to show retinal ganglion cells can be protected with neurotrophic factors. (Yip and So., 2000).

There are many methods for the introduction of genetic material into a cell population. These include transfections or infection of cells outside the body and subsequent implantation, polyethylenimine mediated delivery, and viral vector techniques. Some of these techniques are described in the following papers: Federoff, et al., 1997; Friedmann, 1989; Geddes, et al., 1999; Gokhan, et al., 1998; Kaplitt and Makimura, 1997; Lemkine and Demeneix, 2001; Nakamura, 2001; Nakamura, et al., 2000; Peltekian, et al., 1997; Thorsen and Tysnes, 1997; Trojanowski, et al., 1997; Wax and Patil, 1994; Whittemore and Snyder, 1996; Zlokovic and Apuzzo, 1997a; Zlokovic and Apuzzo, 1997b.

Viral vectors have the added hazard of activating the immune system that can have catastrophic effects on neighboring cells or the entire organism. Accordingly, a method of transfection that circumvents the immunological dangers and inherent inefficiencies involved with the use of virus delivery systems has been developed according to the invention. This method entails the encapsulation of genetic material and/or organelles with biological components that do not induce a significant (or harmful) immune response. One such encapsulant (i.e., cyclodextrin) is already approved by the FDA for human use. A nucleic acid molecule and/or organelle is generally complexed to the encapsulant, enabling the transfection of a specific type of cell or tissue. Once the cell is transfected, the genetic material as an example, can be translated into a functional, necessary protein, if desired. In some embodiments, the transfection efficiency of this method is at least 20%, a rate that has not been duplicated by other non-viral mechanisms.

Cholera toxin is the major virulent factor produced by Vibrio cholerae. It is comprised of one A subunit having ADP-ribosyltransferase activity and five B subunits that function in GM1 receptor binding. (Liljeqvist, et al., 1997; McCann, et al., 1997; Rusnati, et al., 2002). Also Cholrea toxin B subunit (CTB) has been used in humans and has been observed to induce mild immune response that disappears 1 year later when taken through inhalation. (Bergquist, et al., 1997; Qadri, et al., 2000). Internalization of cholera toxin has been reported to involve intracellular trafficking through a retrograde vesicular pathway to the trans-Golgi network, Golgi apparatus and endoplasmic reticulum. This intracellular trafficking may be altered when the cholera toxin B subunit is complexed with cyclodextrin. The complexing and antigen presenting capabilities of cholera and its subunits have been described for vaccines. (Dertzbaugh and Macrina, 1989; Dertzbaugh, et al., 1990; George-Chandy, et al., 2001; Hirabayashi, et al., 1991; Michalek, et al., 1992; Wang, et al., 2001). However CTB has only been described as a tracing molecule in the CNS. (Ling, et al., 1998).

Cyclodextrin is a cyclic polysaccharide that contains naturally occurring D(+)-glucopyranose units in an α-(1,4) linkage. Common forms of cyclodextrin are α-cyclodextrins, β-cyclodextrins, and γ-cyclodextrins which respectively comprise 6, 7 and 8 glucopyranose units. Cyclodextrins have been used as solubilizing agents for pharmaceutical agents. Although not intending to be bound by any particular theory, it is postulated that the solubilizing activity derives from the toroid-like conformation of the cyclodextrin molecule and the hydrophobic cavity formed therein. The size of the cavity is directly related to the number of glucopyranose units per molecule, and as a result, the cavity diameter is estimated to be 5.2, 6.4 and 8.3 angstroms for α-, β- and γ-cyclodextrins, respectively. (Albers and Muller, 1995; Bekers, et al., 1988; Betts, 2001; Hingerty, et al., 1984; Irie and Uekama, 1997; Kondo, et al., 1976; Ohtani, et al., 1989; Okabe, et al., 1993; Sharma and Janis, 1991; Thompson, 1997; Uekama, et al., 1981).

The invention presents a novel method for the non-viral introduction of genetic material and/or organelles into cells either in vitro or in vivo. Such transfection results in the subsequent production of, for example, an encoded protein at levels that are in some instances at least greater than 10% of background levels or those achieved using prior art methods. This discovery has broad implications, particularly for the maintenance or regeneration of neurons.

Methods:

Culture of Primary Cells, Cell Lines, and Tissue or Explant Cultures:

In vitro: Retinal explant culture: The animals were anesthetized with a combination of pentobarbital and valium. The retinal explants were taken from mice that were approximately 9 months of age. The eyes were removed and placed in iced Neuobasal Medium without L-glutamine GibcoBRL 21103-031. The retinas are removed from the pigment epithelium and sectioned. Four retinal slices are placed in a culture well on a semipermeable membrane and allowed to flatten. Each well contains 1.25 ml of culture medium (Neuralbasal Medium with L-glutamine, B27, Asp, Glu and Penn/Strep). Each of the retinas is transfected with EGFP (enhanced green fluorescent protein) plasmid (plasmid size 4.7 kb, Clontech), combined with the delivery vehicle. The tissue is maintained in an incubator at 37° C. under 5% C02 for up to ten days. At the end of each culture period the tissues are fixed with 2% paraformaldehyde for 2 days. The cultures are examined under fluorescent illumination with a Nikon Diaphot inverted microscope. The dark field pictures are taken with a 10× objective, with a DCS620 digital camera.

In vitro: culture of astrocytes has been described in U.S. Pat. No. 6,106,827. Briefly, the method involves harvesting brain tissue from two day old Sprague Dawley rat pups (following sacrifice by decapitation). Brain tissue is placed in CMF-Sal G (calcium magnesium free P-SAL G) in a culture dish on ice (Vilijn et al., Proc Natl Acad Sci USA 85:6551-6555 (19,88)). Striata from ten animals are microdissected to seed approximately 30 (1.5 ml) dishes at 5×10⁵ cells per dish. The tissue is minced with forceps, transferred to a 15 ml sterile conical tube, and the supernatant that remains after momentary settling is used to rinse the culture plate. The tissue is then centrifuged (500-1000 g, 1 minute), the supernatant is aspirated off, and the cells are resuspended in 2 ml of 0.1% trypsin (1.0% Gibco #610-5095AE diluted 1:10 v/v with CMF-Sal G) and allowed to incubate for 30 minutes at 37° C. Incubation is followed by recentrifugation (500-1000 g, 1 minute) and resuspension of the pellet in 2 ml of complete media by gentle trituration until a uniform suspension is observed. The cells are plated at a density ratio of 5×10⁵ cells per 1.5 ml of complete media (swirl gently) on poly-D-lysine (Sigma #P7886, pH 8.5) coated plates (35 mm dish, Falcon #3001) (10⁶/10 ml for 100 mm dish, Falcon #3003) and incubated at 37° C., 100% relative humidity and 5% CO₂, for five to six days. The media is replaced with ice cold media (1.5 ml for 35 mm dish; or 10 ml for 100 mm dish) and the dishes are agitated to remove neural non-adherent cells (Vilijn et al., 1988). Subsequently, the media (37° C.) is changed every 4 to 5 days, until the cells grow to confluency (about two weeks), and then the cells are passaged every 3 weeks using trypsin to release the cells from the poly-D-lysine coated plates. At this point, the cells are either used for transfection or for primary culture experiments.

Identity of the astrocyte cells can be validated by glial fibrillary acidic protein (GFAP) staining and morphology. Astrocytes at low density have star-like shapes and are very flat; at high density they form a “cobble-stone” pattern. Neurons, on the contrary, have long processes (neurofilaments), and are less than 1% of the cells. Fibroblasts look very similar to astrocytes, but are GFAP negative. Oligodendricytes are dark cells with short processes which are much smaller than astrocytes and sit on the surface of the astrocytes. Using the above-described protocol, over 95% of the astrocyte cells were GFAP positive.

Growth of hippocampal neurons can be carried out as described by Shogomori et al. (J. Biol. Chem. 276:9182-9188, 2001.)

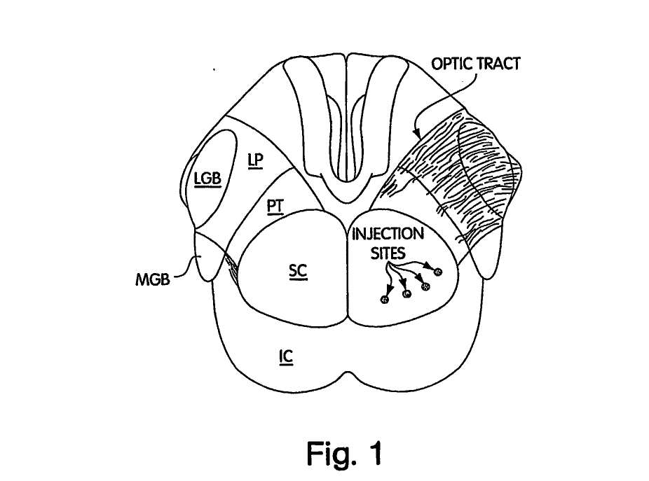

Surgical Techniques: The adult Syrian hamsters were first anesthetized with intraperitoneal injection of valium (50 mg/kg) and nembutal (10 mg/kg). The animal was fitted in the stereotaxic apparatus under complete anesthesia. The cortex was exposed and aspirated to expose the dorsal surface of the SC on the right side. Each animal received four 0.5 microliter injections of transfection material into the right superior colliculus. (See FIG. 1) The surgical opening in the skull was packed with gelfoam and the overlying scalp was sutured. Each animal also received an intra-ocular injection of one microliter of material into the vitreous humor of the right eye. This was done with the aid of a glass micropipette (tip diameter˜10 μm) attached to a General Valve Pico Spritzer. (Mikkelsen, 1992; Ling, 1994, SFN abstract; Ling, et al.1998). The subject was then returned to its cage, under a heat lamp, and monitored until it recovered. The M.I.T. Committee on Animal Care approved all protocols involving the use of live animals.

In vivo tissue preparation: The animals were sacrificed, 4 days after the procedure, with an overdose of anesthesia, perfused transcardially with 0.9% NaC1 and 0.25% NaNO₃ buffer (pH 7.4) followed by 2% paraformaldehyde in 0.1M phosphate buffer (pH 7.4). The brains and eyes were removed and postfixed at 4° C. for four additional days. The brains and eyes were cryoprotected in 30% sucrose at 4° C. until they sank. The tissue was cut on a cryostat in 30-micron coronal sections. These sections were mounted directly on gelatin-coated slides.

Plasmids: The plasmids used herein encode Bcl-2 (plasmid size 6.17 kb), EGFP (plasmid size 4.7 kb), and ECFP (plasmid size 4.7 kb). Plasmid pDSRed is 4.7 kb in length.

In vitro plasmid: The dried cDNA plasmid with an upstream cytomegalovirus (CMV) promoter directing expression of the EGFP transcript was obtained from Clontech (Palo Alto, Calif.). In the methods described herein, the plasmid is encapsulated in a delivery vehicle comprising CTB and/or γ-cyclodextrin.

In vivo plasmid: The dried cDNA plasmid with an upstream CMV promoter directing the expression of the human Bcl-2 transcript was obtained from Upstate Biotechnology (Lake Placid, N.Y.). In the methods described herein, the plasmid is encapsulated in a delivery vehicle comprising CTB and/or γ-cyclodextrin.

Transfection: Cyclodextrin and a cDNA plasmid (in aqueous solution) are mixed at room temperature using a vortex for 2 minutes in an eppendorf tube. Cholera toxin B is added and the mixture is vortexed for another 2 minutes at room temperature. The mixture is then incubated at 4° C. for 2-4 hours, with intermittent mixing. The mixture is then added to a cell culture or tissue section one hour after retinal explants were placed on the culture well membranes (recommended volume of as little as 1 μl). The cultures were then placed in an incubator and removed at 24 hour intervals (for in vitro transfection) or 4 days of exposure (for in vivo transfection), following which cells were harvested and tested for uptake of the plasmid. The cultures were examined under fluorescence illumination with a Nikon Diaphot inverted microscope.

Transfection vehicle preparation: The vehicles used to deliver the genetic material were combinations of Cholera toxin B subunit (CTB) complexed with a plasmid and either β-cyclodextrins or γ-cyclodextrins. Cholera toxin B subunit can be obtained from commercial sources such as List Biological Laboratories, Inc. (Campbell, Calif.), and Calbiochem. Various forms of cyclodextrin are commercially available from sources such as RBI (Natick, Mass.), and Sigma Chemical Co. (St. Louise, Mo.). Dry plasmid (50 μg) was mixed with 150 μl cyclodextrin then mixed 10 minutes in a rocker. 10 μl of CTB stock solution was added to the mixture, and the mixture was then placed in the fridge for 2 hours and then mixed again for seconds just before loading into pipette tip. Fluorospheres (FS) from (Molecular Probes, Inc.) were added to the mixture in half of the samples in order to mark the site of injection. There were six different mixtures used for the preliminary test. All six had the Bcl-2 plasmid. The combinations of plasmid and vehicle were as follows:

TABLE 3
Table 3: This table contains the combinations of materials
used in vivo injections.

| | | |
|---|---|---|
| Bcl-2 + CTB | Bcl-2 + CTB + β-cyclodextrin | Bcl-2 + CTB + γ- |
| | | cyclodextrin |
| Bcl-2 + CTB + FS | Bcl-2 + CTB + β-cyclodextrin + | Bcl-2 + CTB + γ- |
| | FS | cyclodextrin + FS |

(Legend: Bcl-2: Bcl-2 plasmid, CTB: cholera toxin B subunit, FS: fluorospheres).

Immunolabeling of Bcl-2 protein in transfected cells in vivo: The mounted sections were first air dried then washed with PBS (pH 7.4) for three times at 10 min interval and pre-blocked in PBS (pH 7.4) containing 2% triton 100, 2% normal rabbit serum, and 2.5% bovine serum albumin for 30 min at room temperature. The slides were then incubated with mouse anti-human Bcl-2 clone 124 from Upstate (Lake Placid, N.Y.) (1:1000 dilution) +2% Triton-100, 2% normal rabbit serum and 2.5% bovine serum albumin for 48 hr at room temp. Slides were then washed in PBS (pH 7.4) for three times of 10 min each. Sections were incubated with fluorescent goat anti-mouse IgG antibodies Alexa-546 (secondary antibody from Molecular Probes, Inc) (1:200 dilution) for 1 hr 30 min at room temperature in the light protected chamber. Slides were then washed in the PBS (pH 7.4) for 4 to 5 times at 5 min interval. The slides were then cover-slipped with Dabco and visualized through the fluorescence microscope with pictures taken with a Kodak DCS 520 digital camera.

In vitro tissue preparation: At the end of each culture period the explants were fixed after 24, 48, 72, and 96 hours post transfection with 2% paraformaldehyde for 2 days. The cultures were examined under fluorescence illumination with a Nikon Diaphot inverted microscope. The dark-field pictures were taken with a 10× objective, with a DCS620 digital camera.

Results:

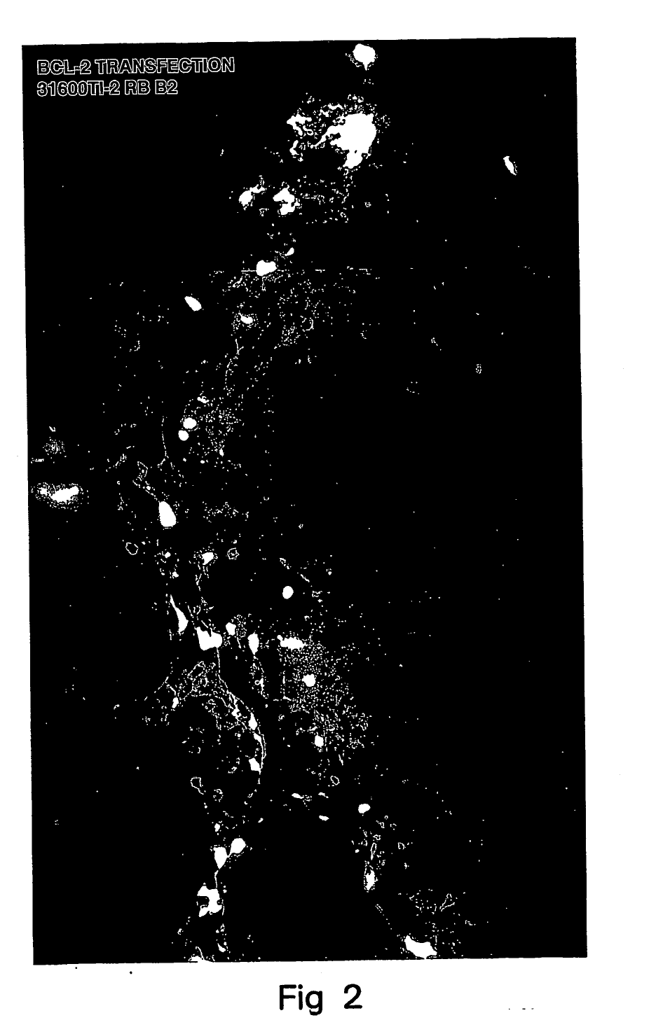

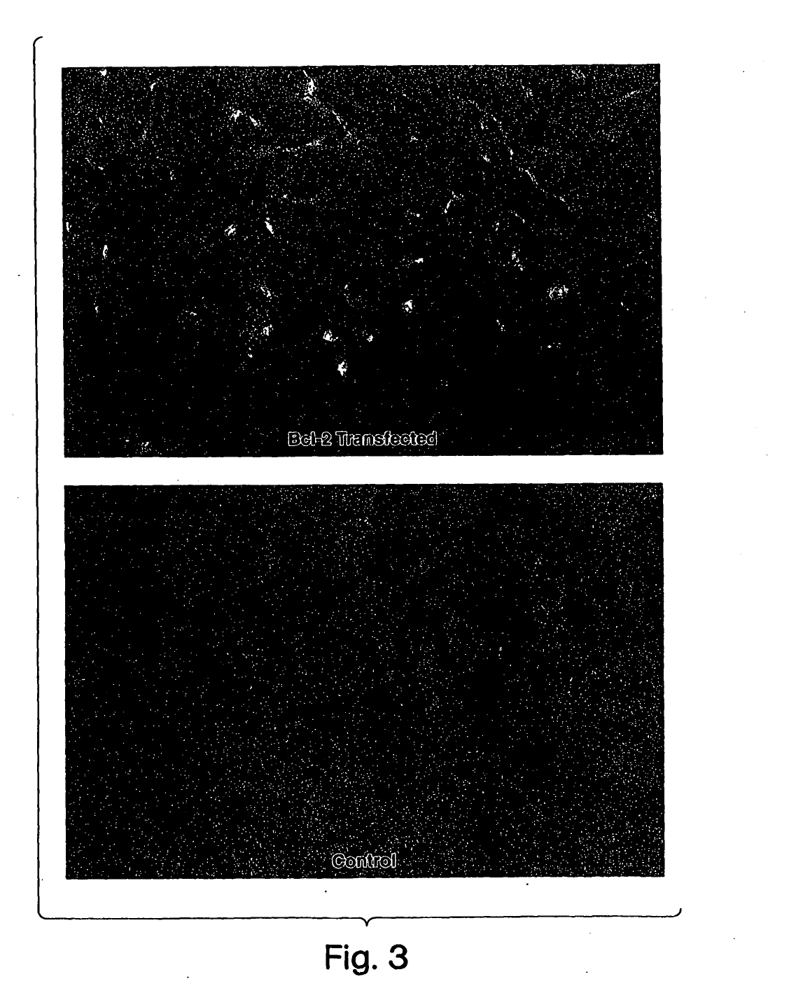

In vivo: The in vivo transfection of the Bcl-2 gene with a resultant production of Bcl-2 protein in the central nervous system (CNS) of adult hamsters has been accomplished using 9-month-old hamsters without viral vectors. The cDNA plasmid encoding Bcl-2 was encapsulated in a delivery vehicle and injected into the caudal midbrain. Visualization of Bcl-2 protein was accomplished by immunohistochemistry using an antibody to the Bcl-2 protein. The antibody was conjugated with an Alexa™ fluorescent marker. The complex of plasmid+CTB+γ-cyclodextrin was found to be the most effective in some instances. The results show that it is possible to transfect the CNS of adult animals without viral vectors and induce production of the encoded protein (FIGS. 2 and 3). This is one of the first successful transfection methods without viral vectors capable of achieving a fairly high transfection rate and production of protein with a single injection in live adult animals.

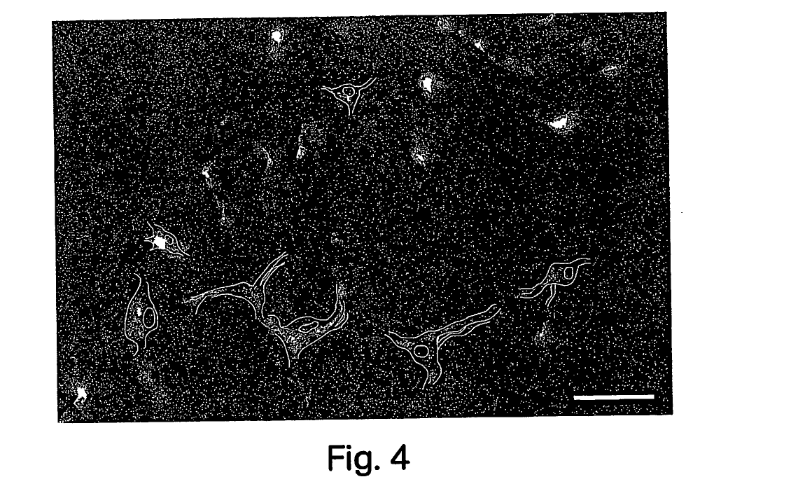

Based on the results the plasmid appears to be retrogradely transported and the Bcl-2 protein is produced. In FIG. 2, the projection from the Zona Inserta shows massive labeling of the Bcl-2 protein. In FIG. 3, the substantia Nigra shows labeling of neurons (top) that project to the SC. The control (bottom panel) is from the contralateral side of the same animal. Absence of staining in the control samples was consistent across all the other cases. There was also faint labeling in the contralateral retina of the successful transfection cases (not shown). In FIG. 4, neurons of the midbrain central gray show Bcl-2 protein that was produced after the transfection with a Bcl-2 plasmid. Note the outlines of some neurons and their nuclei drawn with bright-field microscopy with a reduced aperture to increase contrast. The protein appears to be localized in the cytoplasm of neurons that send direct projections to the superior colliculus (SC).

The best results were obtained from the combination of the delivery vehicle that contained plasmid+CTB+γ-cyclodextrin and the plasmid+CTB+γ-cyclodextrin+FS. The addition of the fluorospheres did not change the effectiveness of the transfection and production of the Bcl-2 protein. Other combinations of delivery vehicle showed no immunoreactivity with the Bcl-2 antibody. To ensure that the results were not gene or allele specific, another gene besides Bcl-2 was tested for the ability to be transfected using the same vehicle. An enhanced green fluorescent protein (EGFP) plasmid was used in the follow up in vitro experiment using mouse retinal explants to confirm the in vivo results.

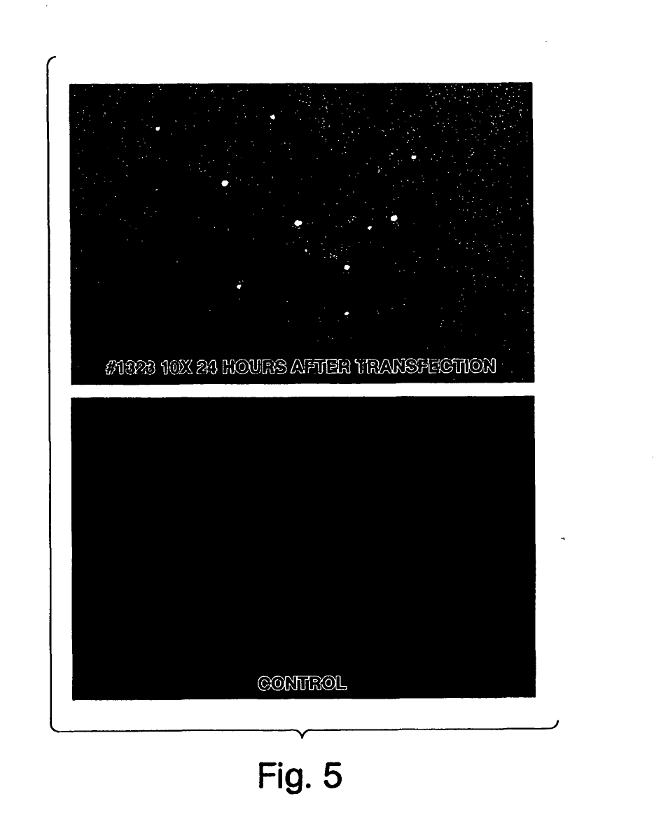

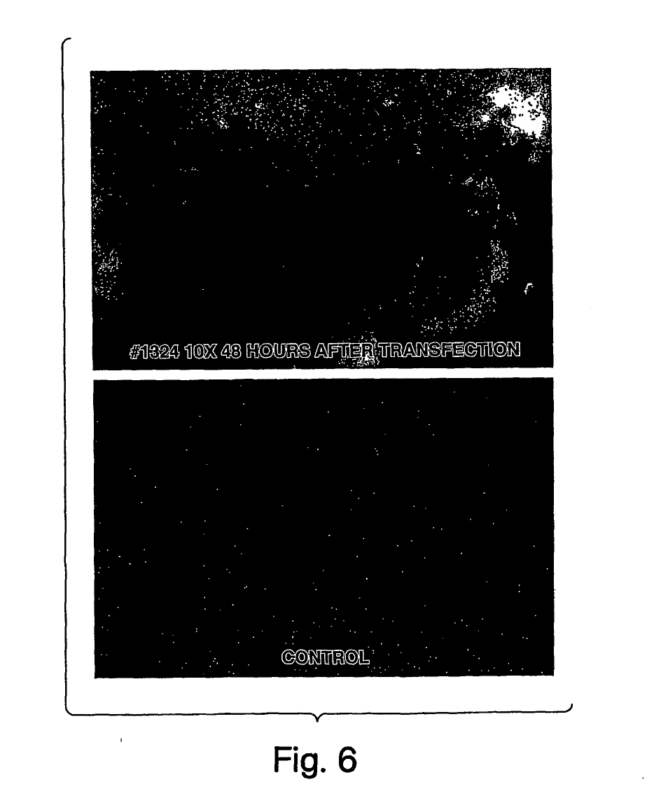

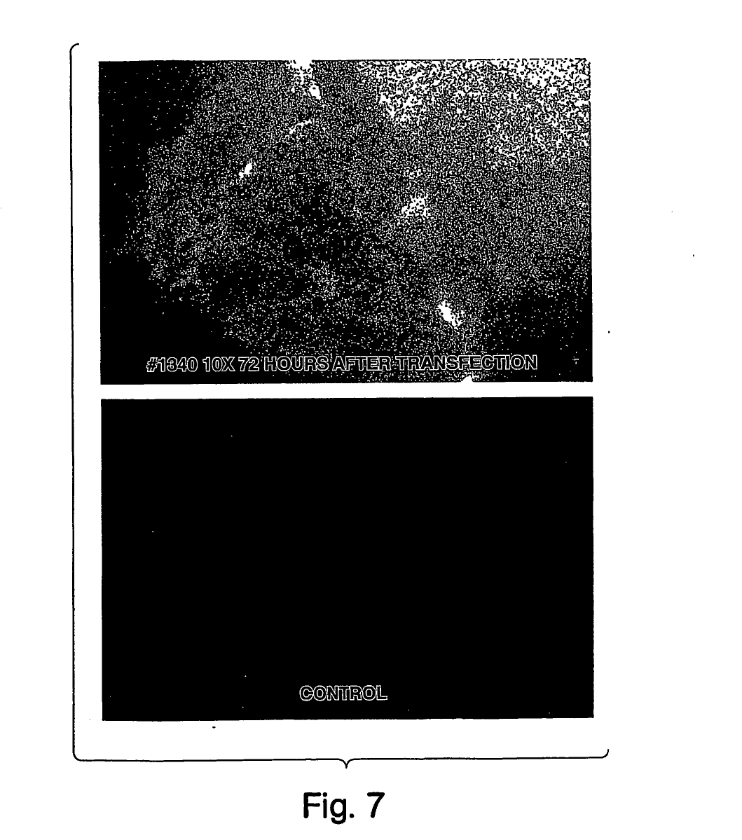

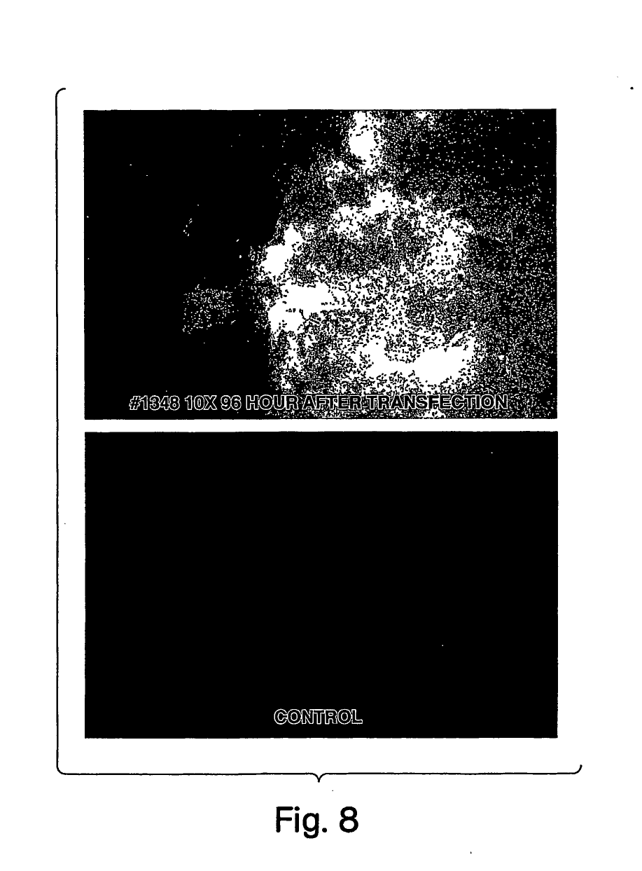

In vitro with EGFP: The confirmation of the transfection technique was proven by the production of EGFP in the transfected explants. Sixty retinal explants from mice ranging in ages from 16 to 28 months were transfected with the delivery vehicle of CTB and γ-cyclodextrin and EGFP plasmid. Twelve controls received the complex with a plasmid lacking the EGFP coding region. The cultures were fixed in twenty-four hour increments at 24, 48, 72, and 96 hours post transfection. All the transfected retinal explant cultures produced EGFP, evidenced by the dark-field pictures taken with a 10× objective, with a DCS620 digital camera. (See FIGS. 5-8.) Each picture set includes a control (bottom panel) that received the same treatment except for the plasmid. The controls were otherwise processed under the same conditions and time. The expression level gradually increases until the entire explant appears to express EGFP.

TABLE 4

| | | MEAN | MEAN LUMINANCE |
|---|---|---|---|
| RETINAL | MEAN | LUMINANCE | INCREASE VS |
| EXPLANTS | LUMINANCE | INCREASE | CONTROL |
| Controls | 35.9 | | |
| 24 hr | 36.71 | 2.26% | 2.26% |
| 48 hr | 49.1 | 33.75% | 36.77% |
| 72 hr | 77.34 | 57.52% | 115.43% |
| 96 hr | 130.52 | 68.76% | 263.57% |

Table 4: The mean luminance for the retinal explants is shown for the controls and transfected explants at each time point. Note the small difference of the control and the explants fixed 24 hrs post transfection. Each of the other time points shows an increase in mean luminance that corresponds to the concentration of EGFP produced. The column to the far right is a comparison of the post-transfected explants to control. There are 12 controls and 60 transfected retinal explants, with 15 for each time point.

Luminance was calculated in adobe Photoshop on raw images. Each picture consisted of 497664 pixels. The mean luminance of the controls was 35.9 and the mean luminance of the 24, 48, 72, and 96 hour retinal explants was 36.7, 49.1, 77.34, and 130.52, respectively. The smallest increase in luminance was shown in the first 24 hours transfection samples. However the other time points show increases from the previous time point (e.g., 33.75% from 24 to 48 hours; 57.52% from the 48 to 72 hours interval; and 68.76% from the 72 to 96 hour timeframe). This shows a significant increase of EGFP expression. The mean luminance percent increases from one time period to another, along with the mean luminance percent difference. The 96 hour retinal explant showed the highest increase in luminance (e.g., a reading of 263.57% when compared to the control at the same time period). Histological results revealed that only the retinal explants transfected exhibited EFGP fluorescence. The controls did not exhibit any fluorescence in the EGFP wavelength. When the controls and the transfected explants are compared, the chi-square was 55, df (4), p<0.001. This demonstrates that these transfections are stable, and that transfections of genes using this non-viral mechanism will produce the desired proteins during the lifespan of the plasmid. The transfection method is not only successful and very efficient, but it can also be utilized for time-lapse expression analysis or transfection treatments in animals. This experiment confirms the in vivo work discussed above.

The transfection technique has proven to be effective in in vivo and in vitro. Once delivered to the cell the nucleic acid molecule can provide a variety of functions, such as promoting or inhibiting the synthesis of additional nucleic acid molecules like RNA molecules, antisense molecules, or polypeptides. The end result of the nucleic acid molecule transfer can be to alter gene expression which in turn can influence cell growth, cell differentiation or cell death, depending upon the nature and function of the encoded polypeptide(s) and the specific application. The nucleic acid molecule can encode one or more native polypeptides that may be absent or defective due to mutation in the wild type gene or from injury.

Discussion:

The results described herein demonstrate successful nucleic acid transfection, in the absence of viral vectors, that achieved a fairly high transfection rate and production of protein with a single injection in adult animals. Delivery of anti-apoptotic genes, proteins, or agents to the affected neurons after injury can be used to enhance neuron survival after injury, thereby facilitating functional regeneration of neurons after injury.

One major advance of this method is the discovery that cholera toxin B subunit can deliver a nucleic acid molecule to a cell of the central nervous system. It has not been recognized previously that cholera toxin B subunit is capable of effecting nucleic acid molecule delivery into cells in vitro or in vivo. With the addition of cyclodextrin, further increases in nucleic acid molecule delivery and expression by recipient cells has been observed, as described herein. Additional compositions and methods of use relating to cholera toxin B subunit and cyclodextrin for the delivery of nucleic acid molecules and/or organelles in vivo, ex vivo and in vitro are also possible.

Retinal ganglion cells have been shown not to grow into the tectum after 10 days of age. Currently, retinal explants are being transfected and grown in culture for the purpose of enhancing regeneration of retinal ganglion cells through the introduction of several genes that have been shown to be important in early development of the visual system. A tissue co-culture can be used to determine the efficiency of transfection along with the effect of introducing these genes in combination in the adult retina.

This method is also a feasible solution for transient or stable transfection of DNA into almost any type of cell, including those that are cultured in vitro. Therefore, the non-viral encapsulated transfection technique can easily be used to replace any of the other currently used methods, such as calcium phosphate precipitation or electroporation.

In summary, the invention provides, in addition to transfection methods and products, methods and products for preserving and regenerating neurons in the CNS. This latter application is particularly important in preserving a neuronal population following injury.

### Equivalents

Those skilled in the art will recognize, or be able to ascertain using no more than routine experimentation, many equivalents to the specific embodiments of the invention described herein. Such equivalents are intended to be encompassed by the following claims.

All references, patents, and patent applications disclosed herein are incorporated by reference in their entirety.

What is claimed is presented below and is followed by a sequence listing that contains sequence information for those sequences listed herein.

We claim:

1. A non-viral delivery complex comprising
a cyclodextrin, and
a cholera toxin B subunit or functional equivalent thereof.

2. The non-viral delivery complex of claim 1, wherein the cyclodextrin is selected from the group consisting of α-cyclodextrin, β-cyclodextrin, and γ-cyclodextrin.

3. The non-viral delivery complex of claim 1, wherein the cyclodextrin is γ-cyclodextrin.

4. The non-viral delivery complex of claim 1, wherein the cholera toxin B subunit or functional equivalent thereof is cholera toxin B subunit or E. coli enterotoxin subunit.

5. The non-viral delivery complex of claim 1, wherein the cholera toxin B subunit or functional equivalent thereof is cholera toxin B subunit.

6. The non-viral delivery complex of claim 1, wherein the cholera toxin B subunit or functional equivalent thereof is a pore or a channel insertion complex.

7. The non-viral delivery complex of claim 6, wherein the channel insertion complex is selected from the group consisting of a chloride channel, potassium channel, sodium channel, calcium channel, or an antiport.

8. The non-viral delivery complex of claim 1, further comprising a biological molecule selected from the group consisting of a nucleic acid molecule and an organelle.

9. The non-viral delivery complex of claim 8, wherein the nucleic acid molecule encodes a polypeptide.

10. The non-viral delivery complex of claim 8, wherein the nucleic acid molecule is an antisense nucleic acid molecule.

11. The non-viral delivery complex of claim 8, wherein the nucleic acid molecule is selected from the group consisting of a DNA molecule, an RNA molecule, and a DNA/RNA hybrid molecule.

12. The non-viral delivery complex of claim 8, wherein the nucleic acid molecule encodes a polypeptide selected from the group consisting of a cystic fibrosis transmembrane conductance regulator (CFTR), a Bcl-2 polypeptide, IFκB, A-beta regulatory protein, parkin, dystrophin, thymidine, kinase, cytosine deaminase, nerve growth factor, apolipoprotein, E ε4, huntingtin, APP, p53, Rb, WT-1, PDGF, lis-1, BDNF, FDGF, IL-12 p40, protease inhibitors and IL-4.

13. The non-viral delivery complex of claim 8, wherein the nucleic acid molecule encodes a cystic fibrosis transmembrane conductance regulator (CFTR).

14. The non-viral delivery complex of claim 8, wherein the nucleic acid molecule comprises a regulatory sequence.

15. The non-viral delivery complex of claim 1, further comprising an organelle targeting compound or a cytoskeleton targeting compound.

16. The non-viral delivery complex of claim 8, wherein the organelle is selected from the group consisting of a mitochondrion, a nucleus, and a chloroplast.

17. A pharmaceutical composition comprising:
an effective amount of the non-viral delivery complex of claim 1, and
a pharmaceutically acceptable carrier.

18.-20. (canceled)

21. A method for delivering a nucleic acid molecule into a cell comprising delivering to a cell a nucleic acid molecule complexed with a cholera toxin B subunit or functional equivalent thereof, in an amount effective to deliver the nucleic acid molecule into the cell.

22.-43. (canceled)

44. A method for producing a non-viral nucleic acid delivery composition comprising
combining a cholera toxin B subunit or a functional equivalent thereof, and a nucleic acid molecule.

45.-46. (canceled)

47. A composition comprising
a cholera toxin B subunit or functional equivalent thereof, and
a nucleic acid molecule.

48.-62. (canceled)

63. A method for delivering an organelle into a cell comprising
delivering to a cell an organelle complexed with a cholera toxin B subunit or functional equivalent thereof, in an amount effective to deliver the organelle into the cell.

64.-80. (canceled)

81. A method for producing a non-viral organelle delivery composition comprising
combining a cholera toxin B subunit or a functional equivalent thereof, and an organelle.

82.-83. (canceled)

84. A composition comprising
a cholera toxin B subunit or functional equivalent thereof, and
an organelle.

85.-94. (canceled)

95. A method for promoting cell survival comprising contacting a cell with an effective amount of an anti-apoptotic agent in a non-viral delivery complex comprising a cholera toxin B subunit or functional equivalent thereof for a time sufficient to transfect the cell.

96.-109. (canceled)
